(19) United States
(12) Patent Application Publication
(10) Pub. No.: US 2005/0267136 A1
(43) Pub. Date: Dec. 1, 2005

(54) Method of inhibiting dihydrofolate reductase; screening assay for the identification of novel therapeutics and their cellular targets

(75) Inventors: Eric D. Brown, Oakville (CA); Xiaoming Li, Burlington (CA); Gerard Wright, Cambridge (CA); Jonathan D. Cechetto, Ancaster (CA); Michela Juran, Hamilton (CA); Rebecca Hartlen, Oakville (CA); Ranjana Pathania, Hamilton (CA)

Correspondence Address: BERESKIN AND PARR, 40 KING STREET WEST, BOX 401, TORONTO, ON, M5H 3Y2, CA

(73) Assignee: McMaster University, Hamilton (CA)

(21) Appl. No.: 11/090,148

(22) Filed: Mar. 28, 2005

Related U.S. Application Data
(63) Continuation-in-part of application No. PCT/CA04/00151, filed on Feb. 4, 2004.
(60) Provisional application No. 60/444,684, filed on Feb. 4, 2003.
(60) Provisional application No. 60/515,644, filed on Oct. 31, 2003.

(51) Int. Cl. A61K 31/517

(52) U.S. Cl. 514/266.4

(57) ABSTRACT

A novel screening assay for identifying therapeutic agents and their cellular targets is described. The assay is useful in developing new antibacterial, antifungal, antiparasitic and anti cancer therapeutics. New inhibitors of dihydrofolate reductase (DHFR) have been identified and their cellular target confirmed using the assay of the present invention. Methods of treating diseases that benefit from an inhibition of DHFR are also described.

### FIELD OF THE INVENTION

The present invention relates to the use of compounds identified as inhibitors of Escherichia coli dihydrofolate reductase (DHFR), the mechanism of action of said compounds being confirmed using an assay of the present invention. The invention also relates to a screening assay for the identification of novel antibacterial, antifungal, antiparasitic and anticancer therapeutics and their cellular targets and to methods of treating diseases using agents identified using the assay.

### BACKGROUND OF THE INVENTION

One of the most significant hurdles in target-based drug discovery is that of the gap between in vitro potency in the inhibition of the function of a protein and efficacy against the target cell. With modern biochemical and medicinal chemical tools of lead generation and lead optimization, drug discoverers can rightly expect that given the appropriate time and resources, very potent compounds can be found for a purified protein target. This is because the physical principles for the development of potency against a protein target are, to a great extent, understood. Potency against the purified target, however, is only one of the requirements of efficacy in target-based drug discovery. A more daunting hurdle, particularly for antimicrobial drug discovery, is the development of compounds that can penetrate living cells to reach intracellular targets. Unlike those principles for the development of potent lead compounds against protein targets, our understanding of natural laws governing access and interaction of a given compound to the intracellular space of a bacterial, fungal or even human cell are poorly understood.

For many years researchers have exploited the effect of gene dosage and protein expression in molecular genetic studies of resistance to chemotherapeutic agents. Such studies have been particularly beneficial to the identification of resistance genes for compounds with antibacterial (1-4), antifungal (5-8), anti-parasitic (9, 10) and anticancer (11, 12) properties. From these studies it is understood that overexpression of a protein target will often lead to resistance to the chemotherapeutic agent owing to two general mechanisms. In one mechanism, perhaps the most common, overexpression of a protein involved in the modification or efflux of the chemotherapeutic agent leads to resistance. Alternatively overexpression of the protein target itself often also leads to resistance. The latter facilitates the identification of genes that are the targets of agents of unknown mechanism.

In view of the foregoing, there is a need in the art to develop screening assays that allow the simultaneous identification of novel antibacterial, antifungal, antiparasitic or anticancer agents and their cellular targets.

### SUMMARY OF THE INVENTION

The present inventors have used a high-throughput in vitro screening assay to identify agents with inhibitory activity against Escherichia coli dihydrofolate reductase (DHFR). Accordingly, the present invention relates to a method of inhibiting DHFR comprising administering to an animal in need thereof, an effective amount of a compound selected from one or more of:
a compound of Formula I;
a compound of Formula II;
any one of compounds 1-11 as shown in Table 1; and
pharmaceutically acceptable salts and hydrates of a compound of Formula I, a compound of formula II and compounds 1-11.

Further, the invention includes the use of a compound selected from one or more of:
a compound of Formula I;
a compound of Formula II;
any one of compounds 1-11 as shown in Table 1; and
pharmaceutically acceptable salts and hydrates of a compound of Formula I, to inhibit DHFR in an animal in need thereof, as well as the use of a compound selected from one or more of:
a compound of Formula I;
a compound of Formula II;
any one of compounds 1-11 as shown in Table 1; and
pharmaceutically acceptable salts and hydrates of a compound of Formula I, to prepare a medicament to inhibit DHFR in an animal in need thereof.

In an embodiment of the invention the DHFR is bacterial DHFR, in particular E. coli DHFR.

The present inventors have also developed a robust system to simultaneously identify potential therapeutic agents and the cellular targets of the agents. The method exploits principles of target overexpression and drug resistance for the development of a high throughput screening method for the identification of the therapeutic agents and their targets. Using this system, it was confirmed that the antibacterial activity of the compounds presented above is related to their capacity to inhibit DHFR.

Accordingly, the present invention provides a method for identifying a candidate therapeutic agent and a cellular target molecule that is modulated by the agent comprising:
(a) contacting a plurality of test agents with a first target cell;
(b) selecting test agents from step (a) that inhibit the growth of the first target cell, wherein said selected test agents are candidate therapeutic agents;
(c) contacting a candidate therapeutic agent identified in step (b) with (i) the first target cell and separately with (ii) a second target cell that overexpresses one or more genes;
(d) comparing the growth of the first target cell with the second target cell wherein the inhibition of growth of the first target cell and not the second target cell indicates that the second target cell overexpresses the cellular target molecule of the candidate therapeutic; and
(e) identifying the cellular target molecule.

The present invention also provides a method for identifying a candidate therapeutic agent and a cellular target molecule that is modulated by the agent comprising:
(a) contacting a candidate therapeutic agent with (i) a first target cell and separately with (ii) a second target cell that overexpresses one or more genes;
(b) comparing the growth of the first target cell with the second target cell wherein the inhibition of growth of the first target cell and not the second target cell indicates that the second target cell overexpresses the cellular target molecule of the candidate therapeutic agent; and
(c) identifying the cellular target molecule.

The present invention also extends to any candidate therapeutic agents and cellular target molecules identified using the above assays.

The present invention also includes a kit for use in identifying candidate therapeutic agents and their cellular targets comprising the first and the second target cells and instructions for the use thereof.

Other features and advantages of the present invention will become apparent from the following detailed description. It should be understood, however, that the detailed description and the specific examples while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will now be described in relation to the drawings in which:

FIG. 1 shows plasmid maps of two different clones selected from the genomic library for ddlA.

FIG. 2 is a graph showing a duplicate screen of 1000 Maybridge compounds for growth inhibition of E. coli MC1061. Statistical analysis of the screening data established a Z-factor (13) of 0.42. A threshold for active molecules of three standard deviations from the mean corresponded to optical density values of 0.37 (indicated by the box in the bottom left corner of the graph). Fifteen actives were identified from this duplicate screen.

FIG. 3 shows a replicate plot of the screen of 50,000 small molecules against E. coli DHFR. Compounds that perturbed E. coli DHFR activity to three standard deviations below the high control mean in both replicates (Hit Zone) were deemed to be active against DHFR, and were selected for secondary screening.

FIG. 4 are graphs showing the IC₅₀ analysis for competitive inhibition. Data are shown for DHFR with a known DHF-competitive inhibitor, Trimethoprim, (panel A) and apparent competitive inhibitor 9 (panel B). Plots for IC₅₀ determination are shown at 30 μM DHF (◯) and 100 μM DHF (●). IC₅₀ values were extracted from assay data using nonlinear regression analysis (SigmaPlot 8.0 software, SPSS Science, Chicago, Ill.) of the equation v=a (1−[I]/(IC₅₀+[I])+c, where v is the reaction rate, a is the amplitude of inhibition, [I] is the inhibitor concentration and c is residual activity at infinite inhibitor concentration.

FIG. 5 is a schematic showing the model of DHFR binding by 6 and 7 (inset) into the E. coli DHFR active site, in the presence of NADPH (PDB code 1RX3) (33). Modeling is based upon the structure of 1 bound to C. albicans DHFR (PDB code 1IA1) (31), and was constructed using SYBYL 6.8 with the Biopolymer module (Tripos Inc., St Louis, Mo.).

FIG. 6 is a graph showing the dependence of the MIC on arabinose concentration for EB492 (strain of E. coli that has pAK01 and pBAD-foIA) in Luria Bertani media. Open circles are tetracycline and closed circles are trimethoprim.

FIG. 7 are graphs showing the dependence of the MIC on arabinose concentration for EB492 in Luria Bertani media. Open circles are tetracycline and closed circles are the molecule indicated.

FIG. 8 is a replicative plot for the test wells of a duplicate screen of 8640 compounds—the primary screen for compounds with growth inhibitory activity. Growth in the test wells is expressed as percentage of that in control wells. A total of 301 actives were identified in the hit zone (gray inset) that was established at a threshold of 75% growth relative to the control wells.

FIG. 9 is a flow chart of the outline of the screening process.

FIG. 10 shows the identification of suppression clones for the growth inhibitory activity of trimethoprim. Shown are petri plates from a proof of principle suppression experiment where 10⁵ cells of E. coli MC1061 were exposed to increasing concentrations of trimethoprim in LB-Str-Amp agar. The cells were from a selection pool, containing a multicopy genomic library cloned into pGEM7, and a control pool, containing empty vector pGEM7. Suppressors were those clones from the library showing resistance not seen in the control pool. These colonies were found to contain identical clones containing DNA coding for the neighboring genes yabF, kefC, and folA at the cloning site of pGEM7.

FIG. 11 is a graphical representation of the regulated suppression of growth inhibition through controlled expression. Shown is the influence of MIC on the controlled expression of DHFR from an arabinose inducible promoter system pBAD18-folA in E. coli CW2553 containing pAK01. (A) The arabinose dependence of the MIC in LB-Amp-Cmp broth of the positive control trimethoprim (open circles) compared to that for the negative control tetracycline (closed circles). (B) Analogous data for compounds 1a (closed circles), 2a (closed triangles), and tetracycline (open circles). The construct pBAD18-dolA was made as follows. Gene folA was amplified by PCR from genomic DNA of E. coli MG1655 using the primers 5′-C GCT CTA GAT TTT TTT TAT CGG GAA ATC TCA ATG-3′ [SEQ ID NO: 1] and 5′-CTA AAG CTT TTA CCG CCG CTC CAG AAT C-3′ [SEQ ID NO: 2], containing XbaI and HindIII restriction sites (underlined), respectively. The resulting product was cloned into the XbaI and HindIII site folA pBAD18-Apr to create pBAD18-folA that puts the expression of gene folA under the control of the arabinose promoter. Controlled arabinose-inducible expression from plasmid pBAD18-folA was accomplished by transformation into E. coli strain CW2553 containing pAK01.

FIG. 12 is a graphical representation of the suppression of growth inhibitory activities of compounds 1a, 2a, and analogs (structures are shown in Table 4) as wells as controls, trimethoprim and ciprofloxacin of E. coli MC1061 clones harboring pGEM7-acrB (black bars) and pGEM7-yabF-kefC-folA (gray bars). Fold suppression for each clone was calculated from MIC determinations for the compound indicated in LB-Str-Amp and was relative to E. coli MC1061 containing pGEM7.

FIG. 13 is a graphical representation of the fold suppression versus the calculated LogP, demonstrating the trend toward suppression by AcrB and hydrophobicity.

FIG. 14 shows the identification of multicopy suppressors using phenotypic growth array. Plate 1 shows array of all the essential genes of E. coli on LB without any drug (control). Plate 2, 3 and 4 show growth of suppressors in the presence of 2, 4 and 8 fold MIC of cycloserine. Schematic representation of gene categories on the phenotypic array is shown in the middle panel. Suppressor genes identified on the array are indicated.

FIG. 15 is a schematic showing the network of suppressors with different antibiotics at medium stringency. Interactions are represented as directional edges extending from the antibiotics. murA, ddl, folA and dxr (highlighted) are the known targets for phosphomycin, cycloserine, trimethoprim and fosmidomycin respectively. Bigger nodes, antibiotics; red nodes, essential genes; small blue nodes, non-essential genes (transporters). The chemical suppressor-interaction network was constructed using osprey visualization tool (Breitkreutz, B. J. et al. Genome Biology (2003) 4 (3), R22. http:f/biodata/mshri.on.ca/osprey).

FIG. 16 is a schematic showing the network of suppressors with different antibiotics at highest stringency. The same network and color scheme is used as for FIG. 15.

FIG. 17 is a graph showing the fold MIC suppression (MIC suppressor/MIC wild type cells) exhibited by yjeE and rpsR in the presence of different antibiotics.

FIG. 18 shows a multicopy suppression array of yeast essential genes from a portion of a library used to identify the target of fluconazole.

FIG. 19 shows a screen of 957 Yeast clones against no drug (control), 32 μg/mL, and 64 μg/mL of fluconazole. The known target ERG11 is seen growing in the upper left of the first plate with fluconazole.

FIG. 20 shows a 1536 array of all 957 Yeast clones on one plate against no drug (control), 32 μg/mL, and 64 μg/mL of fluconazole.

### DETAILED DESCRIPTION OF THE INVENTION

I. Dihydrofolate Reductase Inhibitory Compounds

Dihydrofolate reductase (DHFR) is a well-characterized enzyme (EC 1.5.1.3) that catalyzes the NADPH-dependent reduction of 7,8-dihydrofolate (DHF) to 5,6,7,8-tetrahydrofolate (THF). Tetrahydrofolate is an important cofactor for a number of one carbon transfer reactions and is essential for the biosynthesis of purines, pyrimidines, several amino acids (15). One of the most significant consequences of inhibition of this enzyme is thyrnidylate deficiency leading to the disruption of DNA synthesis. Thus DHFR has long been recognized as a drug target for a wide range of diseases including cancer (16), malaria (17) and bacterial infections (18). Trimethoprim has found particular clinical utility as an inhibitor of DHFR that shows striking selectivity for the bacterial enzymes over that from the human host (19). Clinical resistance to trimethoprim has, however, limited its use to all but a few therapeutic indications (20). A high-throughput screen of Escherichia coli DHFR using a diverse, high-quality library of compounds was performed in order to identify novel inhibitors of the bacterial enzyme.

Using a high throughput screening assay described in greater detail hereinbelow, 11 compounds were identified as competitive inhibitors of DHFR. The structures of the 11 compounds are shown in Table 1. Four of these 11 molecules were evaluated for their antibacterial efficacy against a laboratory strain of E. coli and against the same strain that was overexpressing recombinant E. coli DHFR. The minimum inhibitory concentration (MIC) for all of these molecules showed a dependence on the expression of DHFR in this latter strain, which is consistent with the conclusion that the antibacterial activity of these molecules is related to their capacity to inhibit bacterial DHFR. This is further support for the ability of the assay of the present invention to identify therapeutic agents and cellular target molecules that are modulated by the agents.

In light of the identification of the 11 compounds shown in Table 1 as inhibitors of bacterial DHFR, the present invention further relates to a method of treating conditions that benefit from an inhibition of DHFR, particularly bacterial DHFR, comprising administering to an animal in need thereof, an effective amount of a compound selected from one or more of a compound of Formula I, and pharmaceutically acceptable salts and solvates thereof: 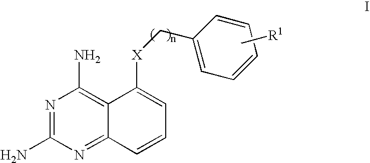
wherein
R¹ is selected from the group consisting of C₁₋₄alkyl, halo and CF₃;
X is O or S; and
n is 0 or 1.

The present invention also relates to the use of a compound selected from a compound of Formula I as defined above, and pharmaceutically acceptable salts and hydrates thereof, to treat conditions that benefit from an inhibition of DHFR, particularly bacterial DHFR, as well as the use of a a compound selected from a compound of Formula I as defined above, and pharmaceutically acceptable salts and hydrates thereof, to prepare a medicament to treat conditions that benefit from an inhibition of DHFR, in particular bacterial DHFR.

The group “R¹” may be located at any position on the phenyl ring. In embodiments of the invention, R¹ is located at the position para to the “X” substituent.

In specific embodiments of the invention, the compound of Formula I is selected from one or more of compounds 1, 2, 3, 4 and 5 as shown in Table 1, and pharmaceutically acceptable salts, solvates or hydrates thereof.

Further the present invention relates to a method of treating conditions that benefit from an inhibition of DHFR, in particular bacterial DHFR, comprising administering to an animal in need thereof, an effective amount of a compound selected from one or more of a compound of Formula II and pharmaceutically acceptable salts and solvates thereof: 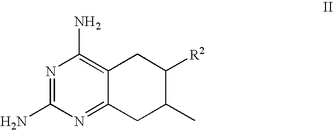
wherein R² is selected from the group consisting of H and C₁₋₄alkyl.

The present invention also relates to the use of a compound selected from one or more of a compound of Formula II as defined above, and pharmaceutically acceptable salts and solves thereof, to treat conditions that benefit from an inhibition of DHFR, in particular bacterial DHFR, as well as the use of an effective amount of a compound selected from one or more of a compound of Formula II as defined above, and pharmaceutically acceptable salts and solvates thereof, to prepare a medicament to treat conditions that benefit from an inhibition of DHFR, in particular bacterial DHFR.

In specific embodiments of the invention, the compound of Formula II is selected from one or more of compounds 6 and 7 as shown in Table 1, and pharmaceutically acceptable salts and solvates thereof.

Finally the present invention further relates to a method of treating conditions that benefit from an inhibition of DHFR, in particular bacterial DHFR, comprising administering to an animal in need thereof, an effective amount a compound selected from one or more of compounds 8 to 11 as shown in Table 1, and pharmaceutically acceptable salts and solvates thereof. In embodiments of the invention the compound is compound 9 as shown in Table 1, and pharmaceutically acceptable salts and solvates thereof.

The present invention also relates to the use of a compound selected from one or more of compounds 8 to 11 as shown in Table 1, and pharmaceutically acceptable salts and solvates thereof, to treat conditions that benefit from an inhibition of DHFR, in particular bacterial DHFR, as well as the use of a compound selected from one or more of compounds 8 to 11 as shown in Table 1, and pharmaceutically acceptable salts and solvates thereof, to prepare a medicament to treat conditions that benefit from an inhibition of DHFR, in particular bacterial DHFR. In embodiments of the invention the compound is compound 9 as shown in Table 1, and pharmaceutically acceptable salts and solvates thereof.

By inhibiting DHFR, the compounds may be used to treat any condition in which inhibition of this enzyme provides a desirable effect, for example, cancer, malaria and bacterial infections.

In particular, the condition that benefits from an inhibition of DHFR is bacterial infection. Accordingly, the present invention also relates to a method of treating bacterial infections comprising administering an effective amount of a compound selected from one or more of:
(a) a compound of Formula I, as defined hereinabove;
(b) a compound of Formula II, as defined herein above;
(c) a compound selected from compounds 8-11 as shown in Table 1; and
(d) pharmaceutically acceptable salts and solvates of (a), (b) and (c),
   to a cell or animal in need thereof. In specific embodiments of the invention, the compound of Formula I is selected from one or more of compounds 1-5 as shown in Table 1 and the compound of Formula II is selected from one or more of compounds 6 and 7 as shown in Table 1.

Further, the present invention includes the use of a compound selected from one or more of:
(a) a compound of Formula I, as defined hereinabove;
(b) a compound of Formula II, as defined herein above;
(c) a compound selected from compounds 8-11 as shown in Table 1; and
(d) pharmaceutically acceptable salts and solvates of (a), (b) and (c),
   to treat bacterial infections, or to prepare a medicament or pharmaceutical composition to treat bacterial infections.

The bacteria may be any bacteria whose growth is affected by the inhibition of DHFR. In an embodiment of the invention the bacteria are, for example, E. coli, Bacillus Subtilis, Streptococci, Staphylococci, Enterococci, Salmonella, Haemophilus influenza, Mycobacterium spp, Pseudomonas aeruginosa, Bacillus anthracis and Helicobacter pylori. In a further embodiment of the invention the bacterial infection is an E. coli infection.

The compounds of Formulae I and II, and compounds 1-11, may also be used as tools, for example, in in vitro screening assays for inhibitors of DHFR, or in any such assay where inhibition of DHFR is desired. In such assays, the compound may be labeled, for example, with a radioactive label or fluorescent label. Accordingly, the present invention also relates to a method of inhibiting DHFR in vitro comprising administering an effective amount of a compound selected from one or more of:
(a) a compound of Formula I, as defined hereinabove;
(b) a compound of Formula II, as defined herein above;
(c) a compound selected from compounds 8-11 as shown in Table. 1; and
(d) salts and solvates of (a), (b) and (c),
   to a cell or assay mixture.

The present invention further relates to the use of a compound selected from one or more of:
(a) a compound of Formula I, as defined hereinabove;
(b) a compound of Formula II, as defined herein above;
(c) a compound selected from compounds 8-11 as shown in Table 1; and
(d) salts and solvates of (a), (b) and (c),
   to inhibit DHFR in vitro.

In embodiments of the invention, the in vitro assay involves bacterial DHFR. In further embodiments the bacteria are for example, E. coli, Bacillus Subtilis, Streptococci, Staphylococci, Enterococci, Salmonella, Haemophilus influenza, Mycobacterium spp, Pseudomonas aeruginosa, Bacillus anthracis and Helicobacter pylori. In a further embodiment of the invention the bacteria are E. coli.

The compounds of Formulae I and II, and compounds 1-11, are either commercially available or may be prepared using standard procedures known to a person skilled in the art. Compounds 1-11 were purchased from Maybridge (Cornwall, England). The structure of compound 9 (Table 1) was incorrectly identified by the company. Exhaustive nuclear magnetic resonance (NMR) experiments have confirmed the structure to be as shown in Table 1. The compound and its biological activity remain the same.

The formation of solvates of these compounds will vary depending on the compound and the solvate. In general, solvates are formed by dissolving the compound in the appropriate solvent and isolating the solvate by cooling or using an antisolvent. The solvate is typically dried or azeotroped under ambient conditions.

The term an “effective amount” or a “sufficient amount” of an agent as used herein is that amount sufficient to effect beneficial or desired results, including clinical results, and, as such, an “effective amount” depends upon the context in which it is being applied. For example, in the context of administering an agent for inhibiting DHFR, an effective amount of an agent is, for example, an amount sufficient to achieve a reduction in DHFR activity as compared to the response obtained without administration of the agent.

As used herein, and as well understood in the art, “treating” or “treatment” is an approach for obtaining beneficial or desired results, including clinical results. Beneficial or desired clinical results can include, but are not limited to, alleviation or amelioration of one or more symptoms or conditions, diminishment of extent of disease, stabilized (i.e. not worsening) state of disease, preventing spread of disease, delay or slowing of disease progression, amelioration or palliation of the disease state, and remission (whether partial or total), whether detectable or undetectable. “Treating” or “treatment” can also mean prolonging survival as compared to expected survival if not receiving treatment.

“Palliating” a disease or disorder means that the extent and/or undesirable clinical manifestations of a disorder or a disease state are lessened and/or time course of the progression is slowed or lengthened, as compared to not treating the disorder. In the context of treating a bacterial infection, palliating may, for example, refer to the inhibition or reduction of the infection.

The “inhibition” or “suppression” or “reduction” of a function or activity, such as bacterial infection, is to reduce the function or activity when compared to otherwise same conditions except for a condition or parameter of interest, or alternatively, as compared to another conditions.

The term “animal” as used herein includes all members of the animal kingdom including human. The animal is preferably a human.

The term “a cell” as used herein includes a plurality of cells. Administering a compound to a cell includes in vivo, ex vivo and in vitro treatment.

The term “C₁₋₄alkyl” as used herein means straight and/or branched chain alkyl groups containing from one to four carbon atoms and includes methyl, ethyl, propyl, isopropyl, t-butyl and the like.

The term “halo” as used herein means halogen and includes chloro, flouro, bromo, iodo and the like.

The term “pharmaceutically acceptable” means compatible with the treatment of animals, in particular, humans.

The term “pharmaceutically acceptable salt” means an acid addition salt which is suitable for or compatible with the treatment of patients.

The term “pharmaceutically acceptable acid addition salt” as used herein means any non-toxic organic or inorganic salt of any base compound of the invention, or any of its intermediates. Basic compounds of the invention that may form an acid addition salt include those having a basic nitrogen, for example NH₂. Illustrative inorganic acids which form suitable salts include hydrochloric, hydrobromic, sulfuric and phosphoric acids, as well as metal salts such as sodium monohydrogen orthophosphate and potassium hydrogen sulfate. Illustrative organic acids that form suitable salts include mono-, di-, and tricarboxylic acids such as glycolic, lactic, pyruvic, malonic, succinic, glutaric, fumaric, malic, tartaric, citric, ascorbic, maleic, benzoic, phenylacetic, cinnamic and salicylic acids, as well as sulfonic acids such as p-toluene sulfonic and methanesulfonic acids. Either the mono or di-acid salts can be formed, and such salts may exist in either a hydrated, solvated or substantially anhydrous form. In general, the acid addition salts of the compounds of the invention are more soluble in water and various hydrophilic organic solvents, and generally demonstrate higher melting points in comparison to their free base forms. The selection of the appropriate salt will be known to one skilled in the art. Other non-pharmaceutically acceptable salts, e.g. oxalates, may be used, for example, in the isolation of the compounds of the invention, for laboratory use, or for subsequent conversion to a pharmaceutically acceptable acid addition salt.

The term “solvate” as used herein means a compound wherein molecules of a suitable solvent are incorporated in the crystal lattice. A suitable solvent is physiologically tolerable at the dosage administered. Examples of suitable solvents are ethanol, water and the like. When water is the solvent, the molecule is referred to as a “hydrate”.

The compounds may be examined for their efficacy in inhibiting DHFR, in particular bacterial DHFR, using any known assay, or, for example, the assay described in Example 2 hereinbelow. The compounds may also be examined for their efficacy in inhibiting bacterial infection using any known assay, for example by monitoring the growth of the bacteria in the presence of the compounds and comparing to controls.

The compounds of Formulae I and II, and compounds 1-11, or salts, hydrates or solvates thereof, are preferably formulated into pharmaceutical compositions for administration to human subjects in a biologically compatible form suitable for administration in vivo.

The compositions comprising an effective amount of compounds of Formula I, Formula II or compounds 1-11, or salts, hydrates or solvates thereof, can be prepared by known methods for the preparation of pharmaceutically acceptable compositions which can be administered to subjects, such that an effective quantity of the active substance is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1985). On this basis, the compositions include, albeit not exclusively, solutions of the substances in association with one or more pharmaceutically acceptable vehicles or diluents, and contained in buffered solutions with a suitable pH and iso-osmotic with the physiological fluids.

In accordance with the methods of the invention, compounds of Formula I, Formula II or compounds 1-11, or salts, hydrates or solvates thereof, may be administered to a subject in a variety of forms depending on the selected route of administration, as will be understood by those skilled in the art. The compounds or compositions may be administered, for example, by oral, parenteral, buccal, sublingual, nasal, rectal, patch, pump or transdermal administration and the pharmaceutical compositions formulated accordingly. Parenteral administration includes intravenous, intraperitoneal, subcutaneous, intramuscular, transepithelial, nasal, intrapulmonary, intrathecal, rectal and topical modes of administration. Parenteral administration may be by continuous infusion over a selected period of time.

Compounds of Formula I, Formula II or compounds 1-11, or salts, hydrates or solvates thereof, may be orally administered, for example, with an inert diluent or with an assimilable edible carrier, or they may be enclosed in hard or soft shell gelatin capsules, or they may be compressed into tablets, or they may be incorporated directly with the food of the diet. For oral therapeutic administration, the compound may be incorporated with excipient and used in the form of ingestible tablets, buccal tablets, troches, capsules, elixirs, suspensions, syrups, wafers, and the like.

Compounds of Formula I, Formula II or compounds 1-11, or salts, hydrates or solvates thereof, may also be administered parenterally or intraperitoneally. Solutions of the compound can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (1990—18th edition) and in The United States Pharmacopeia: The National Formulary (USP 24 NF19) published in 1999.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringability exists.

Compositions for nasal administration may conveniently be formulated as aerosols, drops, gels and powders. Aerosol formulations typically comprise a solution or fine suspension of the active substance in a physiologically acceptable aqueous or non-aqueous solvent and are usually presented in single or multidose quantities in sterile form in a sealed container, which can take the form of a cartridge or refill for use with an atomising device. Alternatively, the sealed container may be a unitary dispensing device-such as a single dose nasal inhaler or an aerosol dispenser fitted with a metering valve which is intended for disposal after use. Where the dosage form comprises an aerosol dispenser, it will contain a propellant which can be a compressed gas such as compressed air or an organic propellant such as fluorochlorohydrocarbon. The aerosol dosage forms can also take the form of a pump-atomizer.

Compositions suitable for buccal or sublingual administration include tablets, lozenges, and pastilles, wherein the active ingredient is formulated with a carrier such as sugar, acacia, tragacanth, or gelatin and glycerine. Compositions for rectal administration are conveniently in the form of suppositories containing a conventional suppository base such as cocoa butter.

The dosage of compounds of Formula I, Formula II or compounds 1-11, or salts, hydrates or solvates thereof, can vary depending on many factors such as the pharmacodynamic properties of the compound, the mode of administration, the age, health and weight of the recipient, the nature and extent of the symptoms, the frequency of the treatment and the type of concurrent treatment, if any, and the clearance rate of the compound in the animal to be treated. One of skill in the art can determine the appropriate dosage based on the above factors. The compounds may be administered initially in a suitable dosage that may be adjusted as required, depending on the clinical response.

Compounds of Formula I, Formula II or compounds 1-11, or salts, hydrates or solvates thereof, can be used alone or in combination with other agents that treat bacterial infections or in combination with other types of DHFR inhibitors.

II. Screening Assay

The present inventors have developed a robust system to simultaneously identify agents with antibacterial activity and the cellular targets of these agents. This method exploits principles of target overexpression and drug resistance for the development of a high throughput screening method for the identification of therapeutic compounds and their targets.

Using the method of the present invention, the present inventors have isolated genes encoding the targets of three antibiotics whose mechanisms are well understood (i.e., murA for fosfomrycin, ddlA for cycloserine, and folA trimethoprim). A preliminary screen of 1000 small molecules revealed 5 drug-like molecules that were growth inhibitory to E. coli and lead to the isolation of a resistance gene for one of those 5 molecules that was a well characterized multidrug efflux transporter (acrB). Further, a second screen of 50,000 small molecules revealed 62 test compounds as inhibitors of E. coli dihydrofolate reductase (DHFR). A secondary screen of these 62 compounds to determine their IC₅₀ values indicated that 11 of these compounds (those claimed in the previous section) had IC₅₀ values that are consistent with competitive inhibition of DHFR. Two of these 11 compounds were screened at growth inhibitory concentrations against cells harbouring a random multicopy genomic library. This screen confirmed that the cellular target for these compounds is DHFR. Further, one of these compounds also produced a clone that contained the gene encoding acrB, the multidrug efflux transporter, therefore confirming that this latter screen can not only identify the cellular target for a particular drug, but also mechanisms of cellular resistance.

Another screen of 8460 small molecules, that were a subset of the 50,000 compounds referred to above, was performed to find compounds that inhibited the growth of E. coli strain MC1061, a hyperpermeable rough lipopolysaccharide mutant at a concentration of 50 μM. An initial selection of 301 compounds was narrowed down to 49 leads by selecting only those that showed complete growth inhibition at 500 μM on rich solid media and through classification by structural formula. These 49 compounds were screened at growth-retarding concentrations against cells harbouring a random multicopy genomic library of E. coli to identify multicopy suppressors of their antibacterial activity. Of the 49 compounds, 33 produced suppressor clones. Clones suppressing the activity of two of the compounds (the same two identified in the previous screen) were found to contain folA, the gene encoding DHFR, while 31 of the remaining compounds resulted in suppressor clones containing the gene acrB, the multidrug efflux transporter. Once again, one of the two compounds producing a clone containing the folA gene, also produced a clone that contained the gene encoding acrB, the multidrug efflux transporter.

The present invention also includes a genome-scale target-based approach for the simultaneous discovery of small molecules that elicit a desired phenotype from cultured cells and the protein targets of those small molecules. In this approach, cells such as bacteria, fungi, yeasts, parasites or animal cells are screened against many compounds to discover those molecules that inhibit growth. Compounds with growth inhibitory activity are then subsequently exposed to pools of these cells overexpressing selected genes of the target organism. If the protein target for such active molecules is present in high copy, growth inhibition is overcome leading ready identification of the target in question. In the target-based, genome scale method, the procedure involves systematic cloning of many or all genes in the target organism to create a pool of overexpressing clones for selection.

The targeted method has several advantages over random genomic libraries. These advantages include the ability to target particular metabolic pathways or categories of protein. In an embodiment, overexpression clones would be generated for all genes in a given genome so that screens against pools of subsets of the all clones could be conducted. For example, pairs of especially novel targets and active molecules could be discovered in differential screens that used pools of clones overexpressing only proteins of unknown function. To discover cell surface targets and their inhibitory molecules, pools containing only overexpression clones for predicted extracellular proteins would be included. Particularly troublesome to the random approach is the problem of genes encoding resistance proteins. Protein pumps capable of removing active molecules from the intracellular space of a cell can lead to the identification, not of protein targets, but of multidrug efflux pumps. In a further embodiment of this approach, therefore, clones overexpressing resistance proteins-from genomic selection pools are excluded. A further advantage of this approach lies in the identification of overexpression clones once they are selected. In the random library approach, clones selected in pools are identified by DNA sequencing which is a relatively slow and serial technique. With a systematic and targeted library of overexpression clones an ordered microarray of plasmid DNA may be used for hybridization analysis and rapid identification of target genes.

Finally, five different classes of antibiotics as well as the antifungal drugs, fluconazole and cymoxanil, were screened at various percentages of their minimum inhibitory growth concentrations, against systematic, non-pooled, ordered overexpression libraries of essential genes in which each clone overexpresses an open reading frame corresponding to an essential gene in for the growth of E. coli and S. cerevisiae, respectively. This screen identified the known targets for these compounds as well as a number of genes of known and unknown function as suppressors.

The screening assay of the present invention is generalizable to a wide variety of systems including the discovery of novel antifungal, antiparasitic and anticancer molecules and their targets.

Accordingly, the present invention provides a method for identifying a candidate therapeutic agent and a cellular target molecule that is modulated by the agent comprising:
(a) contacting a plurality of test agents with a first target cell;
(b) selecting test agents from step (a) that inhibit the growth of the first target cell, wherein said selected test agents are candidate therapeutic agents;
(c) contacting a candidate therapeutic agent identified in step (b) with (i) the first target cell and separately with (ii) a second target cell that overexpresses one or more genes;
(d) comparing the growth of the first target cell with the second target cell wherein the inhibition of growth of the first target cell and not the second target cell indicates that the second target cell overexpresses the cellular target molecule of the candidate therapeutic; and
(e) identifying the cellular target molecule.

The present invention also provides a method for identifying a candidate therapeutic agent and a cellular target molecule that is modulated by the agent comprising:
(a) contacting a candidate therapeutic agent with (i) a first target cell and separately with (ii) a second target cell that overexpresses one or more genes;
(b) comparing the growth of the first target cell with the second target cell wherein the inhibition of growth of the first target cell and not the second target cell indicates that the second target cell overexpresses the cellular target molecule of the candidate therapeutic; and
(c) identifying the cellular target molecule.

The term “a cell” as used herein includes more than one cell or a plurality of cells.

The first target cell can be any cell to which one wishes to generate a therapeutic agent including, but not limited to, bacteria, fungus, parasites and cancer cells. In one embodiment, the target cell is a bacterial target including model organisms such as Escherichia coli and Bacillus subtilis; and pathogens such as Streptococci, Staphylococci, Enterococci, Salmonella, Haemophilus influenza, Mycobacterium spp, Pseudomonas aeruginosa, Bacillus anthracis and Helicobacter pylori. In a further embodiment of the invention the target cell is a yeast cell, for example Saccharaomyces cerevisiae.

In one embodiment of the invention, each test agent in step (a) is administered at a different concentration in order to determine the minimal inhibitory concentration (MIC) of each test agent. Once the inhibitory concentration of an agent is known, such a concentration, or ranges of percentages thereof, can be used in step (c) when contacting the agent with the first and second target cells. For example each test agent may be screened at one or more concentrations ranging from about 0.001 to about 50 times, suitably about 0.5 to about 40 times, more suitably about 1 to about 2 times, their MIC.

The second target cell will be the same type of cell as the first target cell but will be transformed to overexpress at least the open reading frame from one or more genes present in the first target cell. Methods of transforming cells to express or over-express a gene are well known in the art. In one embodiment, the second target cell is transformed with a multicopy random genomic library that will allow the overexpression of all of the genes present in the first target cell. In this embodiment, the identity of the gene or genes being overexpressed in a second target cell suppressing the activity of the candidate therapeutic agent is carried out by obtaining a sample comprising the DNA from that cell and sequencing, for example using PCR and suitable primers, appropriate portions of the DNA to determine the sequence and therefore identity of the gene or genes being overexpressed.

In another embodiment of the present invention, the second target cell may comprise one or more individual overexpressing cell lines or clones derived from an ordered library of genes. Therefore, in another embodiment of the present invention, the second target cell comprises one or more cell types, each overexpressing at least the open reading frame from one particular gene in the genome of the organism from which the cell is taken. A targeted selection of these cell lines may be pooled so that only genes encoding specific proteins are screened. In this embodiment, the identification of the gene being overexpressed in suppressor clones is done using hybridization analysis with an ordered microarray of the plasmid DNA from the organism, which provides rapid identification of target genes. In other words, DNA from suppressor cells is contacted with the ordered array, and the hybridization of the DNA from the suppressor clones with the array is observed using techniques which are known to those skilled in the art. For example, the DNA from the suppressor cells may be labeled with, for example, a fluorescent label and hybridization with the ordered array observed using any known method of detecting fluorescence. Since, in the ordered array of plasmid DNA, the location and identity of each gene is known, one can readily identify the overexpressed gene in the suppressor cell by observing the area or spot on the microarray to which it selectively hybridizes.

In a further embodiment of the present invention, the candidate therapeutic agent is contacted with a plurality of second target cell types, which are in the form of an ordered library of cell lines comprising, for example, at least the open reading frames of genes from the organism. In an embodiment of the invention, the genes are those which are essential to growth and survival of the organism. The growth of each type of cell in the ordered library of second target cells is compared to the growth of the first target cell, wherein the inhibition of growth of the first target cell and not the second target cell indicates that the second target cell type overexpresses the cellular target molecule of the candidate therapeutic agent. It is convenient for the ordered library cells types, each over-expressing a unique gene from the organism, to be in the form of an array or microarray in which the location or identity of each cell type is readily ascertained or ascertainable. As the stringency of the conditions for exposure of candidate therapeutic agent to the second target cells decreases, the number of suppressor cell lines identified may increase. Therefore more suppressor cell lines may be identified when low stringency conditions are used compared to when medium to high stringency conditions are used. The stringency conditions refer to the concentration of candidate therapeutic agent used in the culture media. By “low stringency conditions” it is meant that the concentration of candidate therapeutic agent used is less than or equal to 1×its MIC. By “medium stringency conditions” it is meant that the concentration of candidate therapeutic agent used is between 2× and 4× its MIC. By “high stringency conditions” it is meant that the concentration of candidate therapeutic agent used is greater than 4×its MIC. In an embodiment of the invention, high stringency conditions are used.

In a strategy to avoid selecting for multidrug efflux pumps the second target cells containing the genomic selection pool might be engineered to avoid cloning efflux pumps. In yet another strategy to avoid selecting for multidrug efflux pumps the second target cells containing the genomic selection pool might be derived from sub-pools that have been shown not to contain cell types that overexpress troublesome efflux pumps. The genomic selection pool, for example, may contain some 20,000 cell types in total but is derived from 20 subpools of 1,000 cell types. Each of the subpools could be screened using a test compound known to select for efflux pumps such as acrAB and acrEF. Subpools that are devoid of cell types overexpressing efflux pumps could then be mixed to generate diverse and nearly comprehensive genomic libraries but that do not contain cell types overexpressing efflux pumps.

In yet another embodiment of the invention, the second target cells may also include cell types overexpressing known drug transport or efflux pump genes for the organism. The use of these types of cells allows the determination of whether or not a candidate therapeutic agent will be a substrate for a drug transport or efflux pump mechanism in the cell. If the candidate therapeutic agent is a substrate for a drug transport or efflux pump mechanism in the cell, the efficacy of the agent may be decreased. This is important information in the development of effective therapeutic strategies that require the presence of the therapeutic agent inside the cell.

The test agents that may be used can be any agent which one wishes to test including, but not limited to, proteins, peptides, nucleic acids (including RNA, DNA, antisense oligonucleotide, peptide nucleic acids), carbohydrates, organic compounds, natural products, library extracts, and other samples that one wishes to test for therapeutic activity against a particular target. In one embodiment, the test agents are from a small molecule library.

The method is adaptable to high-throughput screening applications. For example, a high-throughput screening assay may be used which comprises any of the methods according to the invention wherein aliquots of the target cells are exposed to a plurality of test compounds within different wells of a multi-well plate. The method of the invention may be “miniaturized” in an assay system through any acceptable method of miniaturization, including but not limited to multi-well plates, such as 24, 48, 96 or 384-wells per plate, micro-chips or slides. The assay may be reduced in size to be conducted on a micro-chip support, advantageously involving smaller amounts of reagent and other materials. Any miniaturization of the process which is conducive to high-throughput screening is within the scope of the invention.

III. Uses of the Assay

The present invention includes all possible uses of the screening assay of the invention, some of which are summarized below.

(a) Therapeutic Agents and Targets

The invention extends to any agents or targets identified using the screening method of the invention. Once a potential therapeutic agent is identified using the screening method of the invention, one of skill in the art can readily conduct further tests to prove the therapeutic potential of the agent. One can also further study the targets to further elucidate their role in the disease process.

The invention also includes a pharmaceutical composition comprising a therapeutic agent identified using the screening method of the invention in admixture with a suitable diluent or carrier. The invention further includes a method of preparing a pharmaceutical composition for use in therapy comprising mixing a therapeutic agent identified according to the screening assay of the invention with a suitable diluent or carrier.

(b) Kits

The development of the screening assay of the invention allows the preparation of kits for use in identifying novel therapeutic agents and their targets. The kits would comprise the reagents suitable for carrying out the methods of the invention, packaged into suitable containers and providing the necessary instructions for use. For example, the kit may comprise both the first and the second target cells for use in the assay of the invention. In the specific screen of the invention, the kit may contain a plurality of target cells, each overexpressing a particular gene product(s). The kit may provide instructions for preparing the appropriate target cells as well as instructions for carrying out the assay of the invention.

The term “instructions” or “instructions for use” typically includes a description describing the reagent concentration or at least one assay method parameter such as the relative amount of the reagent-sample admixtures, temperature, conditions and the like.

Accordingly, the present invention provides a kit for use in identifying a therapeutic agent and its cellular target comprising a first target cell to which one wishes to generate a therapeutic agent and a second target cell that overexpresses one or more genes present in the first target cell.

(c) Therapeutic Uses

The assay and kit of the invention allow the identification of novel therapeutic agents that may be used in developing drugs for treating or preventing many diseases and conditions. Such diseases and conditions include, but are not limited to, bacterial, parasitic and fungal infections as well as cancer. Accordingly, the present invention also provides a method of treating a disease comprising administering an effective amount of a therapeutic agent isolated according to the method of the invention to an animal in need thereof.

The term “effective amount” as used herein is defined as an amount effective, at dosages and for periods of time necessary to achieve the desired result. The effective amount of a compound of the invention may vary according to factors such as the disease state, age, sex, and weight of the animal. Dosage regima may be adjusted to provide the optimum therapeutic response. For example, several divided doses may be administered daily or the dose may be proportionally reduced as indicated by the exigencies of the therapeutic situation.

The term “animal” as used herein includes all members of the animal kingdom, including humans. Preferably, the animal to be treated is a human.

(d) Drug Discovery

The present invention also includes all business applications of the screening assay of the invention including conducting a drug discovery business.

Accordingly, the present invention also provides a method of conducting a drug discovery business comprising:
(a) providing one or more assay systems for identifying a potential therapeutic agent;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

In certain embodiments, the subject method can also include a step of establishing a distribution system for distributing the pharmaceutical preparation for sale, and may optionally include establishing a sales group for marketing the pharmaceutical preparation.

The present invention also provides a method of conducting a target discovery business comprising:
(a) providing one or more assay systems for identifying a potential therapeutic agent;
(b) (optionally) conducting therapeutic profiling of agents identified in step (a) for efficacy and toxicity in animals; and
(c) licensing, to a third party, the rights for further drug development and/or sales for agents identified in step (a), or analogs thereof.

The following non-limiting examples are illustrative of the present invention:

### IV. EXAMPLES

### Example 1

In the example described here, the inventors the principles of target overexpression and drug resistance was exploited in the development of a high throughput screening method for the identification of antibacterial compounds and their targets. The method is generalizable to a wide variety of systems including antifungal, antiparasitc and anticancer drug discovery.

General Method

The method begins with the identification of compounds in a small molecule screening library that have antibacterial activity against E. coli strain MC1061 a hyper-permeable rough lipopolysaccharide mutant (21A hyper-permeable strain of E. coli was chosen to maximize the opportunity to detect small molecules with antibacterial potential. Each of the compounds that demonstrated antibacterial activity were subsequently subjected to MIC analysis to determine the minimum concentration necessary to inhibit bacterial growth. Having established growth-retarding concentrations of each active molecule a search for the cellular target of the antibacterial compound was made using a multicopy genomic library of E. coli that was transformed into strain MC1061. Selections for growth from this pool of clones on growth-inhibitory concentrations of an active compound selected for clones that overexpress the target protein. Indeed, a proof of principle was outlined in this Example with the identification in the genomic library of three celebrated bacterial targets using their respective drugs. This approach was also applied to a small commercial library of screening compounds in a preliminary application of the method.

Random Genomic Library from E. coli

The random E. coli genomic library was constructed by Deborah Siegele at Texas A&M University. The library was made by cloning approximately 3 to 4 kb gel-purified fragments from a partial Sau3AI digest of DNA from MG1655. The fragments were cloned into the BamHI site of pGEM7. The library was acquired in the form of a ligation mix that was subsequently transformed into E. coli strain MC1061 (hsdR mcrB araD139 D(araABC-leu)7679 ΔlacX74 galU galK rpsL thi) by electroporation and plated on LB agar selecting for streptomycin (ST, 50 μg/mL) and ampicillin (AP, 50 μg/mL). Some 20,000 colonies were then tooth-picked from these plates after overnight growth (37° C.) such that each clone was transferred to a single well in a 96-well plate containing 200 μL LB-ST-AP broth and grown overnight with shaking at 37° C. Each overnight culture (125 μL) was transferred to a well in a deep-well polypropylene “stock plate” containing 125 μL of 30% glycerol in LB broth. The stock plate was sealed and stored at −80° C. Each overnight culture (50 μL) was also mixed with an equal volume of 30% glycerol in LB broth and transferred to a “screening pool” such that the pool contained all 20,000 clones. The “screening pool” was stored in aliquots at −80° C. and is, as the name suggests, the pool from which the inventors attempt to identify resistance clones. The “stock plate” is a source of the each of the clones for future use.

Proof of Principle with Known Antibiotics.

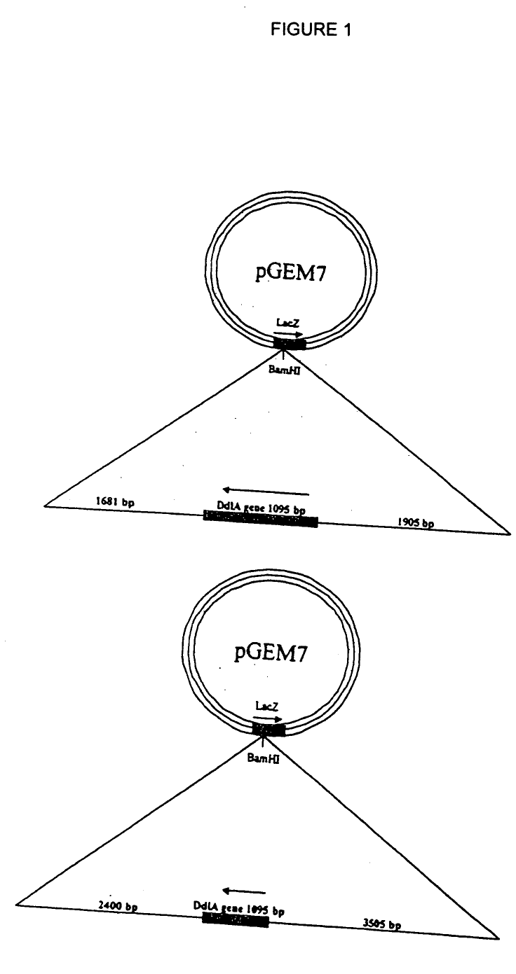

As proof of principle selection for the genes encoding the targets of three well-known antibiotics using the expression screening method was attempted. In a typical experiment untransformed MC1061 and MC1061 transformed with the genomic library (the screening pool) were systematically exposed to increasing concentrations of the following three antibiotics: fosfomycin, trimethoprim and D-cycloserine. More precisely, E. coli strain MC1061 was grown overnight in broth LB-ST, diluted 10³-fold in LB-ST and about 10⁴ bacteria were plated (100 μL per plate) on LB agar-ST with increasing concentrations (0.1, 0.25, 0.5, 1, 2, 4, 8, 16, 32, 64, 128, 256, 512 μg/mL) of each of the antibiotics. In parallel a similar number of bacteria (10⁴) from the screening pool were plated on such plates. In each case colonies were isolated from the screening pool that were resistant to concentrations of these antibiotics that were lethal to MC1061. Table 2 summarizes the results for each of these where 7, 20 and 8 clones were isolated that were resistant to growth inhibitory concentrations of fosfomycin (320 μg/mL), trimethoprim (2.5 μg/mL) and D-cycloserine (160 μg/mL). PCR was used to test for the presence of the expected target gene in the plasmids carried by a subset of these and it was determined that 3 of 7 clones resistant to fosfomycin harbored a plasmid that contained the gene murA. For trimethoprim, 5 of 5 clones contained folA in high copy and for D-cycloserine 6 of 6 contained ddlA. Sequence analysis of the inserts into pGEM7 confirmed for all those tested that the expected target gene was present in clones selected for using each of these antibiotics. FIG. 1 shows the maps of two different clones identified that contained the gene ddlA.

A First Screen Against Library of 1000 Compounds.

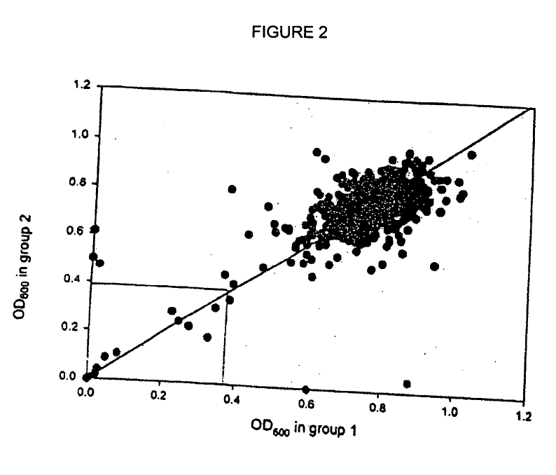

In addition to proof of principle work using well-characterized antibiotics a preliminary screen against a 1000 compound, non-proprietary commercial library from Maybridge (Cornwall, England) was also done. First the identification of compounds with activity against E. coli by screening for inhibition of growth was done. E. coli strain MC1061 was grown overnight in broth LB with streptomycin (ST, 50 μg/mL), diluted 10⁷-fold in LB-ST and deposited into 96-well microwell plates (200 μL/well). To each test well 10 μL of screening compound (1 mM in DMSO) was added and the plate incubated for 36 hours at 37° C. with shaking (150 rpm) before reading the optical density (600 nm). FIG. 2 shows the results of duplicate screens of MC1061 against these 1000 compounds. The result of this duplicate screen was the identification of 15 compounds with statistically significant antibacterial activity.

Of the 15 compounds, five that appeared to be most drug-like (22) and might be reasonable leads for a medicinal chemistry program (Table 2) were focused on. These compounds were re-acquired from the supplier (Maybridge) in quantities necessary for retesting and minimum inhibitory concentration (MIC) determination. MIC testing was on LB-ST agar. E. coli strain MC1061 was grown overnight in broth LB-ST, diluted 103-fold in LB-ST and plated about 104 bacteria (100 μL per plate) on LB agar-ST with increasing concentrations of the compounds in Table 3. In parallel, a similar number of bacteria (10⁴) from the screening pool were plated on another set of plates. For compound SEW04978 3 clones were isolated from the screening pool that were resistant to a concentration of 64 μg/mL while a concentration of 32 μg/mL was lethal to MC1061. The plasmids from these three clones were subsequently purified and sequenced to determine the gene content of the inserts in the respective cloning sites. The plasmid inserts of these three clones proved to be identical and contained the complete open reading frame for a single gene acrB. Gene acrB encodes the acridine resistance pump, a protein that has been well characterized as a multidrug resistance efflux pump (23, 24). The antibacterial action of SEW04978 is presumably abrogated by the overexpression of this cellular pump from the high copy plasmid pGEM7 resulting in resistance to an otherwise toxic molecule.

In summary it is believed that the isolation of a multidrug efflux transporter in this preliminary assay of just 1000 molecules further proves the principle that resistance genes can be isolated for novel antibacterial agents. It is expected that targets of these agents will also be among those resistance genes isolated as was demonstrated for the three antibiotics of known mechanism.

### Example 2

### Screening for Inhibitors of Bacterial DHFR

A library of compounds (50,000) sourced from Maybridge (Cornwall, England) were screened against recombinant E. coli DHFR in a highly automated format. The gene (folA) encoding dihydrofolate reductase (DHFR) was PCR amplified from E. coli MG1655 chromosomal DNA with primers, ^{5′}-C ATC TTA CAT ATG ATC AGT CTG ATT GCG GC-^{3′} [SEQ ID NO: 3] and ^{5′}-CTA CTC GAG CCG CCG CTC CAG MT CT-^{3′} [SEQ ID NO: 4], containing NdeI and XhoI restriction sites (underlined), respectively. The gene was cloned lacking a stop codon into NdeI and XhoI digested pET26b to form pET26b-folA, which incorporates a C-terminal polyhistidine-tag. Polyhistidine-tagged DHFR was purified to homogeneity as described previously (25).

DHF reductase activity was assayed continuously in 96-well microplates by monitoring the decrease of NADPH at an absorbance of 340 nm (26). Assays were carried out at 25° C. and performed in duplicate. The 200 μL reaction mixture contained 40 μM NADPH, 30 μM DHF, 5 nM DHFR, 50 mM Tris (pH 7.5), 0.01% (w/v) Triton and 10 mM β-mercaptoethanol. Test compounds from the screening library were added to the reaction before initiation by enzyme and at a final concentration of 10 μM. High activity controls consisted of reaction mixtures with DMSO only and low activity controls contained 1.5 μM Trimethoprim. Automation for high throughput screening included assay reagent handling in 96 well format. Compound addition, assay monitoring and plate handling were performed using a Sagian-Beckman Coulter linear track with a Biomek FX liquid handler and SpectraMax Plus plate reader (Molecular Devices, Sunnyvale, Calif., USA) integrated into Core Assay System (Beckman Coulter, Mississauga, Ontario, Canada). ActivityBase (IDBS Inc., Emeryville, Calif.), SARgen (IDBS Inc., Emeryville, Calif.) and Spotfire DecisionSite (Spotfire, Inc., Somerville, Mass.) were used for data analysis.

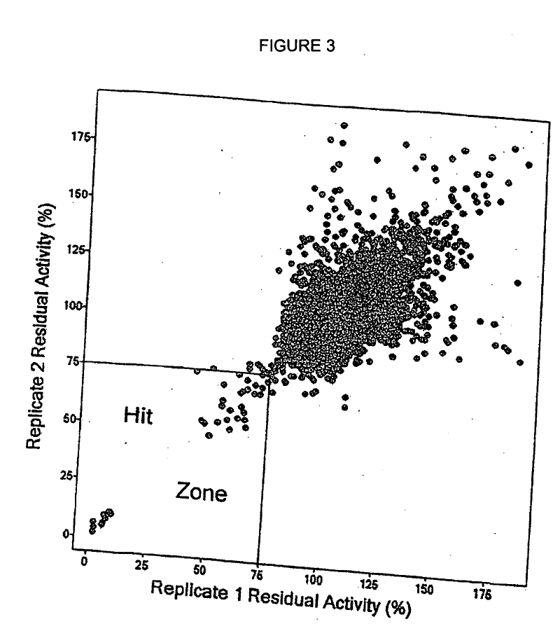

The assay data were of high quality with Z and Z′ statistical scores (27) of 0.66 and 0.73, indicative of good signal to noise in the compound and control wells, respectively. FIG. 3 shows a plot of the screening replicates against one another and illustrates the quality of the entire screen, where absolute replicates would lie on a perfect diagonal. All data are reported as percent residual activity relative to the average of the high controls. Active molecules were identified as those showing less than 75% residual activity, a statistical cut-off three standard deviations below the high control mean. Using this threshold 62 compounds were found to be inhibitors of DHFR, giving a primary hit rate of 0.12% over the entire screen.

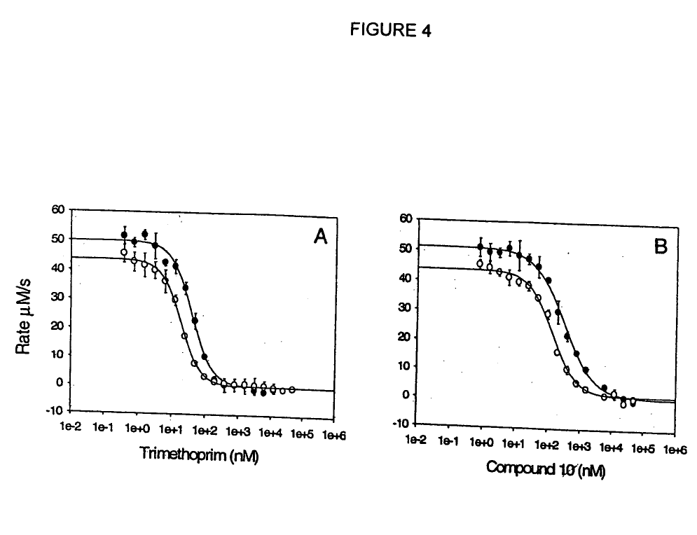

In secondary screening, IC₅₀ determinations were performed for actives from primary screening. This potency analysis was done at two DHF concentrations, 30 μM and 100 μM, to identify compounds that were competitive with DHF. FIG. 4 illustrates sample IC₅₀ curves for trimethoprim, a known inhibitor competitive with DHF (28), and the active compound 9. The ratio of the two calculated IC₅₀ values at the two concentrations of DHF was used to evaluate the competitiveness of each primary hit with respect to DHF. Using the equation IC₅₀=Kᵢ (1+[S]/Kₘ) (29), where Kₘ, DHF=9.5 μM (data not shown), the IC₅₀ ratio of a true competitive inhibitor should equal 2.8 for determinations at 100 and 30 mM DHF. This secondary screen identified 11 inhibitors of DHFR, out of the 62 actives from primary screening, having IC₅₀ ratios consistent with competitive behavior. Table 1 shows the structures of these 11 molecules along with their IC₅₀ data. Compounds 1, 2, 4, 9 and 11 from Table 1 have IC₅₀ values that were in the nanomolar range and those for the remainder of the compounds were in the micromolar range.

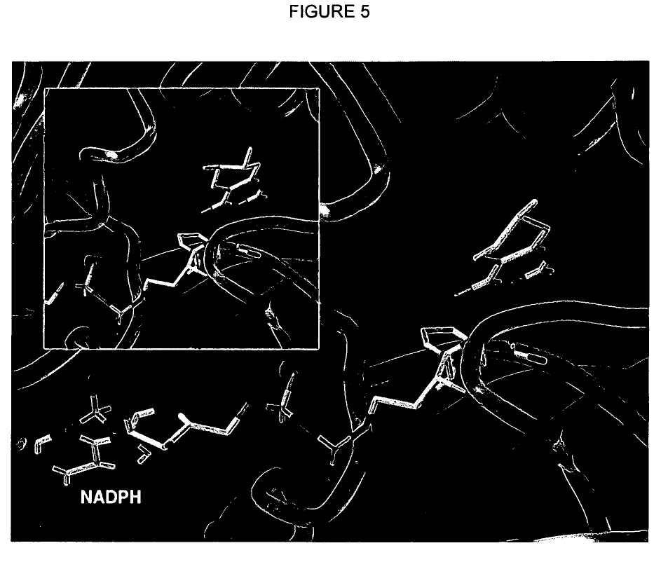

Of the identified compounds the 5-arylthioquinazolines (1 and 2) have been previously identified as active against Candida albicans DHFR (30) but not bacterial DHFR. The structure of 1 bound to C. albicans DHFR has been published (31). Compounds 1-5 have been previously reported as modulators of serine/threonine protein kinase function (32). Based on the structure of DHFR with 1 present in the active site of C. albicans DHFR (31), compounds 6 and 7 were modeled into the active site of E. coli DHFR. Each of these compounds nicely occupies the DHF binding pocket (FIG. 5). Of the remaining molecules, 9 and 11 are novel inhibitors of DHFR that have guanidine in common and are remarkably potent. The structure of compound 9 was incorrectly identified by the commercial sounds. Extensive NMR experiments confirm that the structure is that shown in Table 1. Compound 10 is a quinolinone not previously reported to be active against dihydrofolate reductase.

### Example 3

### Antibacterial Properties of Novel DHFR Inhibitors and Mechanism of Action

Four molecules identified as inhibitors of E. coli DHFR in Example 2 were evaluated for their antibacterial efficacy against a laboratory strain of E. coli and against the same strain that was overexpressing recombinant E. coli DHFR. All of these molecules showed a dependence of minimum inhibitory concentration (MIC) on the expression of DHFR in this strain.

A strain of E. coli in which the expression of the folA gene and ultimately the copy number of the FolA protein could be varied was created for these studies. The folA gene was cloned into the pBAD18-Apr vector (34) and transformed into E. coli strain CW2553 contains the pAKO1 plasmid (35). Strain CW2553 is devoid of a functional chromosomal arabinose transporter while plasmid pAK01 encodes araE, an arabinose transporter under control of a tac (IPTG-inducible) promoter. This system allowed for the controlled expression of the folA gene.

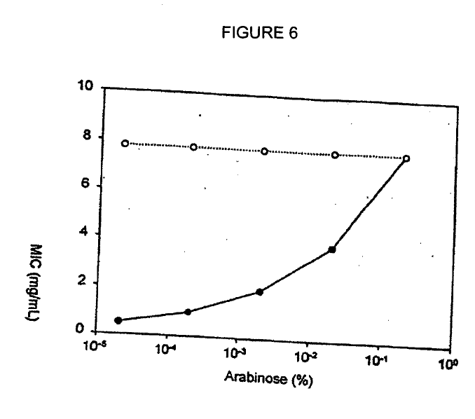

The gene (folA) encoding dihydrofolate reductase (DHFR) was PCR amplified from E. coli MG1655 chromosomal DNA with primers, ^{5′}-C GCT CTA GAT TTT TTT TAT CGG GM ATC TCA ATG-³′ [SEQ ID NO: 1] and ⁵′-CTA AAG CTT TTA CCG CCG CTC CAG AAT C-^{3′} [SEQ ID NO 2], containing XbaI and HindIII restriction sites (underlined), respectively. The resulting PCR product was cloned into the XbaI and HindIII site of pBAD18-Ap^{r} to create pBAD18-folA that puts the expression of gene folA under the control of the arabinose promoter. Plasmid pBAD18-folA was subsequently transformed into CW2553 containing pAK01 to produce E. coli strain EB492. Strain EB492 was systematically exposed to test compounds and to arabinose in order to determine if there was a dependence for the MIC of each test compound on the arabinose concentration in the media. FIG. 6 demonstrates the arabinose dependence of the MIC for trimethoprim, an antibacterial drug understood to target DHFR. Increasing expression of DHFR with increasing arabinose concentration leads to resistance to trimethoprim and not to tetracycline.

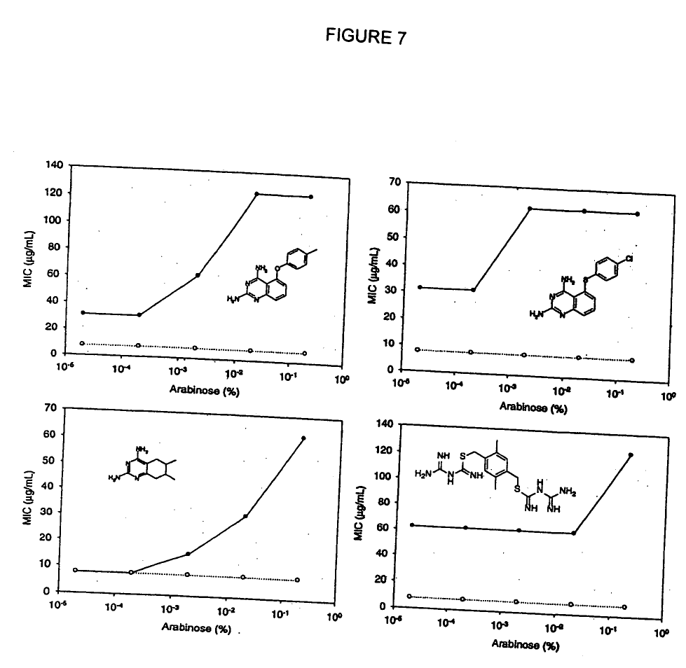

Using this system the mechanism of antibacterial action of 4 molecules identified in the biochemical screen of DHFR in Example 2 was investigated (FIG. 7). In each case these molecules show a dependence of the observed MIC for EB492 on the concentration of arabinose in the media. Such a dependence is consistent the conclusion that the antibacterial activity of these molecules is related to their capacity to inhibit bacterial DHFR.

### Example 4

Two molecules (compound 4 and compound 7, Table 1) identified as inhibitors of DHFR in Example 2 and as growth inhibitory to E. coli in Example 3, were subjected to a search for the cellular target of the antibacterial compounds using the pool of clones harbouring the random multicopy genomic library detailed in Example 1. Exposure of this pool of clones to growth-inhibitory concentrations of these compounds resulted in the selection of clones containing the gene folA, encoding dihydrofolate reductase, for each of the compounds. This outcome is consistent with hypothesis that the cellular target of these antibacterial molecules is DHFR. Furthermore, compound 4 also produced clones that contained the gene acrB, encoding the multidrug efflux transporter. The latter result confirms that the expression screening method is capable of producing both the cellular target of an antibacterial compound add resistance genes such as acrB.

### Example 5

### Multicopy Suppressors for Novel Antibacterial Compounds Reveal Targets and Drug Efflux Susceptibility

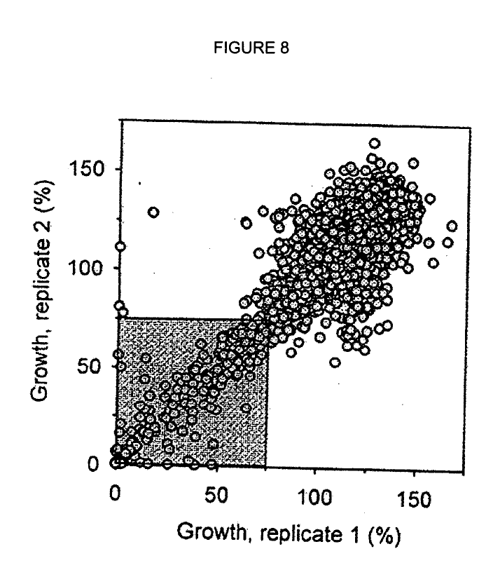

In this experiment, the present inventors have used a 8460 compound library, a subset of the compound library described above. A high-throughput screen was performed to identify compounds that had growth inhibitory activity against E. coli strain MC1061, a hyperpermeable rough lipopolysaccharide mutant, at a concentration of 50 μM in rich liquid media. FIG. 8 shows a replicate plot of the screening data where the quality of the screen is evident in the high correspondence of duplicate determinations. The Z′ statistical values of 0.78 and 0.73 were calculated for replicates 1 and 2, respectively, indicative of a high quality screen. Molecules that demonstrated 25% growth inhibition were judged to be hits in the primary screen resulting in the selection of 301 compounds (3.5% hit rate) for further analysis.

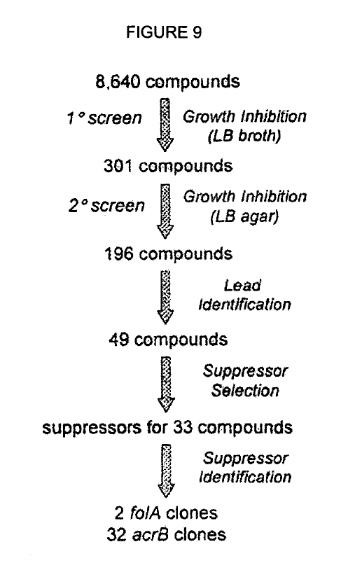

FIG. 9 summarizes the approach to arrive at a small number of growth inhibitory leads for multicopy suppression and subsequent follow up. Of the 301 compounds selected from primary screening, 196 showed complete growth inhibition on rich solid media at 500 μM. This secondary screen was necessarily on solid media and at a higher concentration to test for complete inhibition of growth because of the nature of downstream multicopy suppression experiments. Those experiments required the selection and isolation of clones resistant to otherwise lethal concentrations of the leads and where the MIC of the leads were frequently in the range of 50-500 μM. Classification of these 196 molecules according to structural features facilitated the selection of a representative subset of 49 leads for further analysis. Subsequent MIC analysis on these 49 leads revealed the minimum concentration necessary to inhibit bacterial growth for each active molecule. Having established growth-retarding concentrations of each novel molecule, selection for multicopy suppressors was performed using a pool of clones harboring a multicopy genomic library of E. coli.

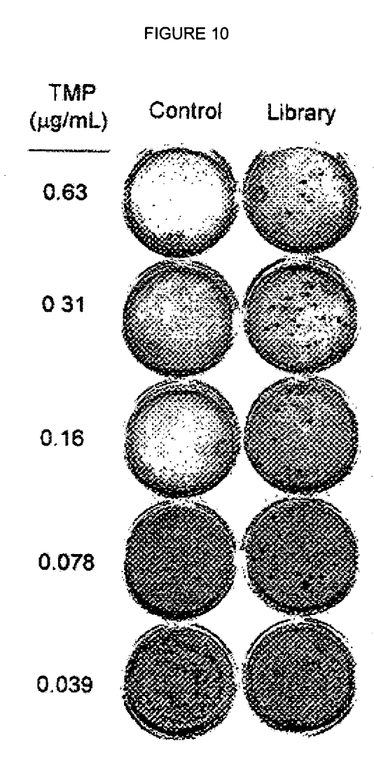

As proof of principle, suppressors of the activity of three well-known antibiotics were first selected. FIG. 10 shows a typical experiment where a control strain and a pool of clones harboring the genomic library were systematically exposed to increasing concentration of the antibiotic trimethoprim. While growth inhibition was seen in the control at 0.078 μg/mL of trimethoprim, suppressor clones were evident at almost ten times that concentration (0.64 μg/mL). Similarly, suppressors were isolated for growth inhibition by cycloserine and fosfomycin (Table 4). Sequencing of the plasmid DNA contained in these clones revealed inserts in the cloning site of pGEM7 corresponding to genomic fragments encoding the targets of these three antibiotics. Trimethoprim produced clones with the fragment yabF-kefC-folA where folA encoding dihydrofolate reductase is the target of trimethoprim. Fosfomycin yielded the fragment ispB-sfsB-murA-yrbA where murA encodes its target UDP-N-acetylglucosamine enolpyruvyltransferase. Cycloserine yielded two different clones containing the fragments yaiW-yaiY-YaiZ-ddlA-yaiB-phoA-psiF and yaiY-YaiZ-ddlA-yaiB where ddlA encodes the target D-ala-D-ala ligase.

Of the 49 leads selected from the screen, suppressor clones could be isolated for 33 of the compounds, while the remainder of the compounds simply did not produce resistant clones. Clones suppressing the activity of two of the compounds, 7 and 4, were found to contain folA encoding DHFR (Table 4). While all of the suppressors isolated for the activity of 7 contained the gene folA, two different suppressors were isolated for 4 at similar frequency, either folA or acrB, encoding the membrane component of the acridine efflux pump. All of the suppressors that were isolated for the remaining 31 molecules contained a clone with a single open reading frame for acrB in the cloning site of pGEM7.

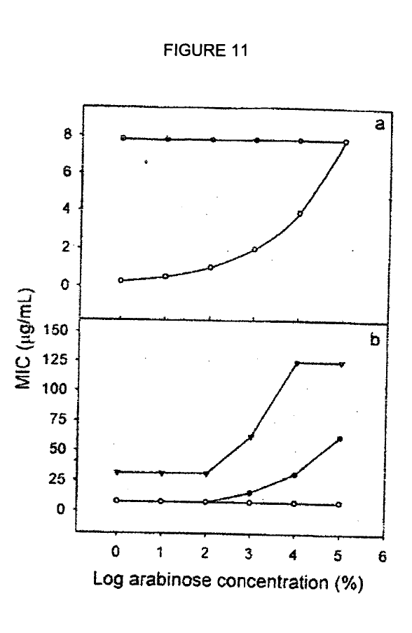

To further confirm that the antibacterial activities of compounds 7 and 4 were related to inhibition of DHFR, folA was subcloned into an arabinose inducible expression system, vector pBAD18 (Guzman et al. (1995) J. Bacteriol. 177, 4121-4130) and this plasmid was placed in an E. coli host, strain CW2553 containing pAK01, that allowed for incremental control of protein expression (Morgan-Kiss et al. (2002) PNAS 99, 7373-7377). With this system, increasing inducer concentrations led to a steady increase in MIC for the control molecule trimethoprim without any impact on the MIC for tetracycline (FIG. 11A). Similarly, the MIC values for 7 and 4 demonstrated an inducer dependence (FIG. 11B) that was consistent with the conclusion that growth inhibition by these molecules was due to inhibition of DHFR.

Compounds 7 and 4 can be broadly classified as 2,4-diaminopyrimidine- and 2,4-diaminoquinazoline-containing molecules, respectively, where trimethoprim, falls into the former structural class. The discovery of these molecules is reported in Example 4 above. In this example, the present inventors have sampled analogs of 7 and 4 from the compound library that were previously identified as inhibitors of DHFR activity and characterized these for their MIC and for their kinetic inhibition constants (Table 5). The latter involved a systematic analysis of the steady-state kinetic behavior of DHFR at a range of dihydrofolate and inhibitor concentrations to reveal competitive inhibition mechanisms for all of the diaminopyrimidines (including trimethoprim) and diaminoquinazolines (data not shown). The diaminopyrimidines 7 and 6 differ by only a methyl group, have comparable Kᵢ values (1.0 and 1.1 μM, respectively), and showed a 4-fold difference in MIC (16 and 64 μg/mL). Likewise, the MIC values of the diaminoquinazolines 4, 3, 1, and 2 did not track with Kᵢ. For example, 4 and 2 differ in the bridging position Y (O or S, respectively) and in the phenyl substitution Z (CH₃ and Cl, respectively) and demonstrated a greater than 6-fold difference in affinity (230 and 38 nM, respectively) but had identical MIC values of 4 μg/mL. The lack of correspondence of MIC and Kᵢ values, despite minor variations in structure, is illustrative of a common difficulty in antimicrobial research of translating gains in biochemical inhibition into increased cellular potency.

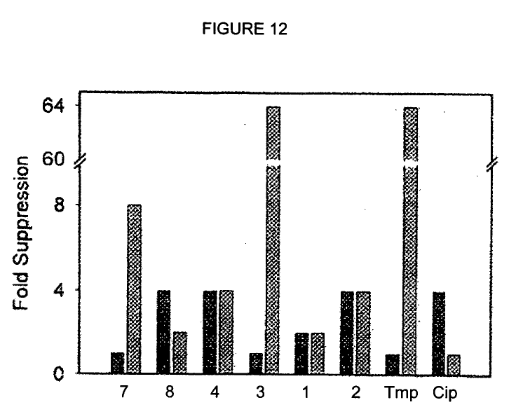

The suppression of the activity of compounds 7, 4 and analogs by clones containing acrB and folA was compared. FIG. 12 shows the fold suppression of the activity of these molecules by clones containing pGEM7-acrB and pGEM7-yabF-kefC-folA relative to a clone containing empty vector pGEM7. The controls trimethoprim and ciprofloxacin showed selective suppression as expected by clones encoding folA and acrB, respectively. Suppression of the activity of ciprofloxacin, for example, was 4-fold by the pGEM7-acrB-containing clone, consistent with a well-documented role for acrB in fluoroquinolone resistance. There was no suppression by pGEM7-acrB and 8-fold suppression with the folA-containing clone. Compound 6, on the other hand, differs only by a methyl group and showed a 4-fold suppression by acrB. The diaminoquinazoline 4, showed an equal propensity to produce clones encoding folA and acrB in the multicopy suppression selection. Compound 4 likewise demonstrated a 4-fold suppression with clones containing either pGEM7-yabF-kefC-folA or pGEM7-acrB, and similar behavior was evident with the diaminoquinazoline analogs 1 and 2. In contrast, the acrB-containing clone showed no capacity for suppressing the activity of analog 3 while demonstrating 64-fold suppression by folA at high copy.

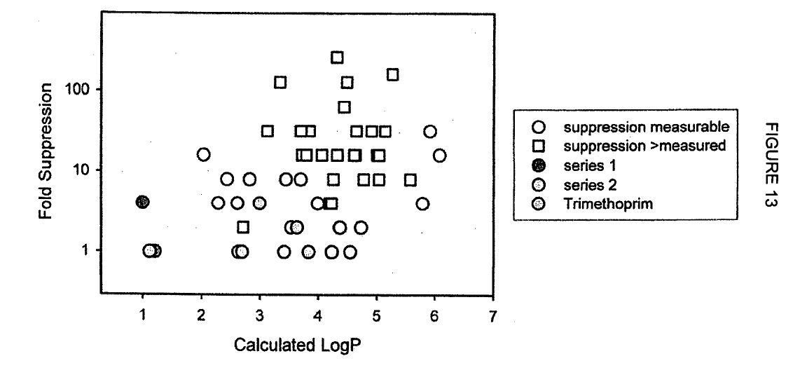

Examination of the fold suppression of growth inhibition by acrB at high copy (pGEM7-acrB) for all 49 growth inhibitory molecules showed a positive correlation between fold suppression and calculated LogP, a commonly used molecular descriptor for general hydrophobicity (FIG. 13).

Materials and Methods:

Primary Screening: The screen of Escherichia coli MC1061 (hsdR mcrB araD139 D(araABC-leu)7679 ΔlacX74 galU galk rpsL thi) against 8640 small molecules was fully automated with the use of a SAGIAN Core System (Beckman Coulter, Inc. Fullerton, Calif.) equipped with an ORCA arm for labware transportation, a Biomek FX with a 96-channel head for liquid handling, and a Spectromax absorbance plate reader (Molecular Devices Corp., Sunnyvale, Calif.); the entire system was integrated through SAMI software (v. 3.5, Beckman Coulter, Inc.). Incubations were done in duplicate and contained 50 μM library compounds sourced from Maybridge plc (Cornwall, UK). E. coli MC1061 was grown overnight in Luria-Bertani (LB) broth containing 50 μg/mL streptomycin (Str), diluted 10⁵-fold in LB-Str broth, and deposited into 96-well microwell plate (200 μl/well). To each test well, 10 μl of screening compound (1 mM in DMSO) was added and the plate incubated for 16 hr at 37° C. with shaking (150 rpm) before reading the optical density (600 nm). High control wells contained 10 μl of DMSO while the low control wells contained 50 μg/ml Ampicillin (Amp). Primary hits were defined as those compounds that reduced growth by 25% compared to the high controls. Activity Base (v. 5.0.5, ID Business Solutions Limited, Emeryville, Calif.), SARgen (v. 1.0, ID Business Solutions Limited), and Spotfire DecisionSite (v. 7.1.1, Spotfire Inc., Somerville, Mass.) were used for data analysis.

Secondary Screen and Lead Selection: Hits from primary screening in liquid media were further analyzed in duplicate for growth inhibition on solid media. E. coli MC1061 were grown overnight in LB-Str broth, diluted 10⁵-fold, and 10 μl/well added into 96-well plates containing LB-Str agar (200 μl/well). Test wells contained compounds at 500 μM (10 μl of 5 mM compound in DMSO). High control wells contained 5% DMSO and low control wells, 50 μg/ml Amp. Plates were incubated for 16 hr at 37° C. before reading by visual inspection. Compounds causing complete growth inhibition were clustered by chemical group functionality and representative compounds were selected as leads for selection experiments using multicopy suppression.

Multicopy Suppression: A random E. coli genomic library was a generous gift of Deborah Siegele (Texas A&M University). The library was in the form of a ligation mix, derived from a partial Sau3AI digest (3-4 kb gel-purified fragments) of DNA from strain MG1655 cloned into the BamHI site of pGEM7 (Promega, Madison, Wis.), and was transformed into E. coli strain MC1061 and plated on LB-Str-Amp agar. Some 20,000 colonies were picked from these plates after overnight growth (37° C.), transferred to a single well in a 96-well plate containing 200 μl LB-Str-Amp broth, and grown overnight with shaking at 37° C. Overnight cultures were pooled, mixed with an equal volume of 30% glycerol in LB broth, and stored in aliquots at −80° C. The latter was referred to as the selection pool. A control pool, E. coli strain MC1061 containing pGEM7, was also grown overnight in broth LB-Str and was subsequently mixed with an equal volume of 30% glycerol in LB broth and was stored in aliquots at −80° C.

Suppressor clones capable of growth in the presence of inhibitory concentrations of compounds were selected using the following procedure. Typically, 10⁵ bacteria from the control and selection pools were plated on LB-Str agar with increasing concentrations of a given compound. Plasmid DNA was prepared from clones in the selection pool that were capable of growth at concentrations that were inhibitory to the control pool. Because many of the suppressor clones were found to contain acrB, encoding the membrane spanning subunit of the acridine efflux transporter, PCR was typically used to screen plasmid DNA derived from suppressors prior to sequencing. This PCR screen employed the forward primer (5′-ATGCTCCTCTAGACTCGAGGMTT-3′) [SEQ ID NO: 5] that annealed to the plasmid and a primer designed to anneal to acrB (5′-TCMTGATGATCGACAGTATGGCT-3′ [SEQ ID NO: 6]). Plasmid DNA from PCR-negative clones were sequenced to determine the cloned insert using the pGEM 7 forward and reverse (5′GAATACTCMGCTATGCATCCMC-3′ [SEQ ID NO: 7]) primers. Nucleotide-nucleotide BLAST was used to determine the portion of genomic DNA cloned into pGEM7.

Minimum Inhibitory Concentration Determination: Determinations of minimum inhibitory concentration were made to characterize growth inhibition of test and control compounds as well as to e=establish the degree of suppression by particular clones selected from the genomic library. Typically, 10⁵ E. coli MC1061 cells were exposed to 2-fold dilutions of the compound from a stock solution of 6.4 mg/ml in DMSO. Incubations were in LB broth using 96-well microwell plates (200 μl/well) for 16 hr at 37° C. with shaking (150 rpm) before determining the optical density (600 nm). Concentrations where the optical density was less than 0.1 absorbance units were deemed MIC.

Determination of Kinetic Inhibition Constants: Recombinant E. coli DHFR was prepared and assayed as described previously. NADPH was constant at 80 μM and dihydrofolate was varied from 10 to 300 μM. Data were analyzed using SigmaPlot version 8.0 software and fit to the Michaelis-Menten equation for competitive inhibition: V=Vₘₐₓ×[S]/K_{M} s (1+I/Kᵢ)+S).

### Example 6

### Screening for High Copy Suppressors of Chemical Lethality using a Growth Array of Overexpressors of Essential Genes

A systematic ordered overexpression library of essential genes of E. coli in which each clone overexpresses an open reading frame corresponding to an essential gene in a multicopy plasmid pCA24N was used for the phenotypic screens. All the essential genes were categorized into four broad functional categories based on COG database (Tatusov, R. L. et al. Science (1997) 278, 631-637, www.ncbi.nlm.nih.gov/COG/). Briefly, minimum inhibitory concentration (MIC) of nine antibioitics belonging to five different categories (cell wall inhibitors: cycloserine and phosphomycin; cell membrane inhibitor: polymyxin; protein synthesis inhibitors: tetracycline, spectinomycin, streptomycin; DNA synthesis inhibitor: norfloxacin; Metabolic inhibitors: trimethoprim and fosmidomycin) were determined against E. coli carrying pCA24N alone (Genobase database ecoli.aist-nara.ac.ip). The essential gene library of E. coli was screened against these antibiotics at ten different concentrations ranging from 0.0625 fold MIC to 32 fold MIC of each antibiotic corresponding to low, medium and high stringency of drug action. The screen was done in the form of 384 well density growth array on LB agar and analyzed for the appearance of resistant/suppressor phenotype in the form of colony forming units.

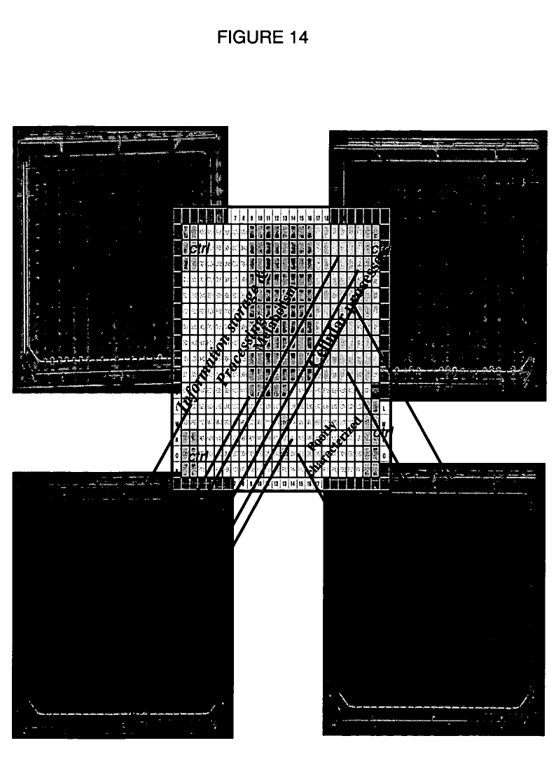

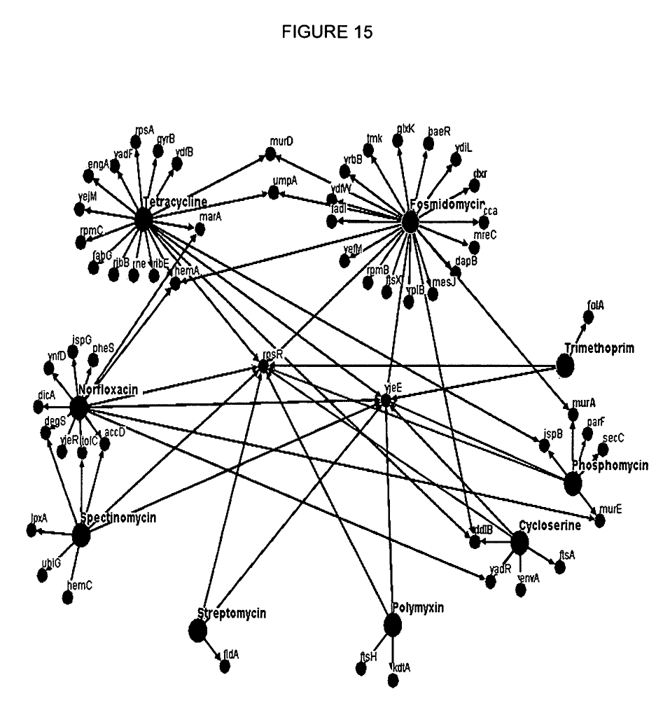

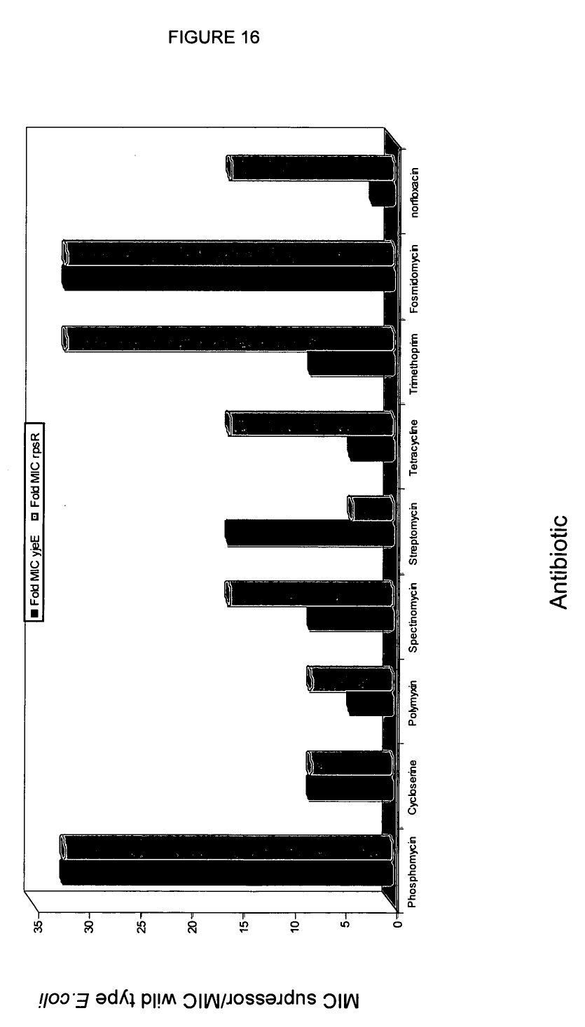

The phenotypic growth array against the five classes of antibiotics not only yielded their celebrated targets but also many genes of known and unknown function as suppressors. The suppressors isolated for cycloserine at low, medium and high stringency are shown in the form of phenoytypic arrays in FIG. 14. The chemical interaction networks of all nine antibiotics and their suppressors isolated on growth arrays were constructed at low (not shown), medium (FIG. 15) and high stringency (FIG. 16).

The genes isolated in response to cycloserine at medium stringency belong to at least 3 different functional categories including the genes of unknown function (FIGS. 14 and 15). This indicates that cycloserine interacts with rpsR (30S ribosomal protein), ftsA (cell division protein), envA (cell envelope protein), yadR, yjeE (proteins of unknown function) and ddI (D-ala-D-ala ligase, the known target) at medium stringency but the interaction is limited to ddI, ftsA and rpsR at high stringency (FIG. 15) and to ddI at the highest stringency (FIG. 16).

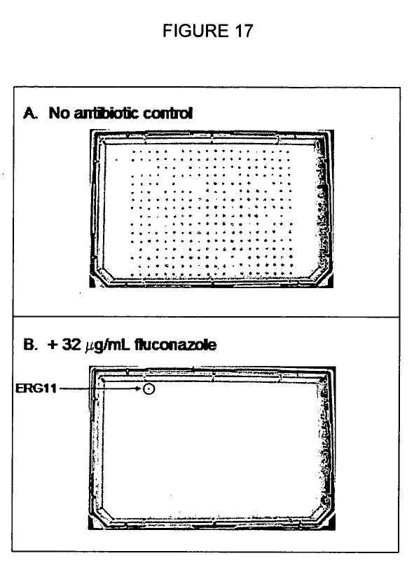

Two suppressors carrying rpsR and yjeE were isolated repeatedly in response to all the drugs at different levels of stringency. The fold MIC of the two suppressors of different antibiotics is compared with the control E. coli cells in FIG. 17.

### Example 7

### Multicopy Suppression Arrays of Yeast Essential Genes Identify the Target of Fluconazole

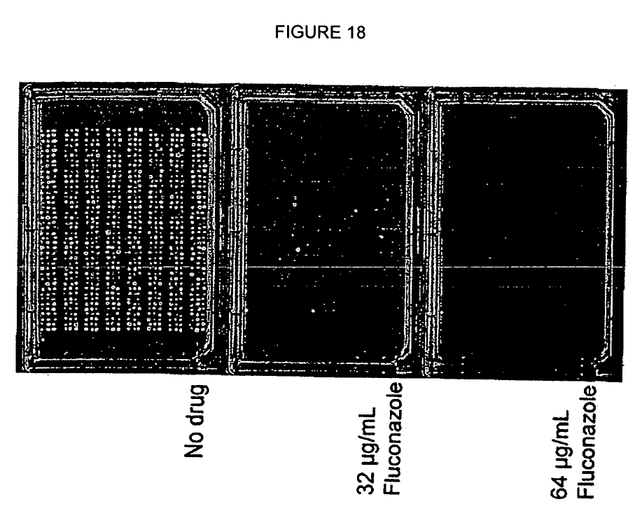

An ordered library of essential fungal (the yeast Saccharomyces cerevisiae) genes that can be selectively overexpressed was constructed resulting in an increase in copy number of the associated protein. The plasmid used was pGAL_DEST_cFLAG_STOP. FIG. 18 shows a portion of the yeast library of essential genes pinned in 384 format onto solid agar and demonstrates the utility of the multicopy suppression approach in identifying the target (ERG11) of the antifungal drug fluconazole.

Approximately 1100 essential yeast genes have been the focus of the yeast ordered library. Each gene was cloned under the control of the inducible Gal promoter, cloned first into E. coli, then S. cerivisiae.

To determine the MIC of compounds versus S. cerevisiae DL1 (His, Leu, Ura)/pGAL, 96 well plates of SC-Leu, 2% glucose with frozen YEC (yeast essential clones) library, grown for 48 hours at 30° C. Shallow well compound agar plates with concentrations of compound of 0.5, 0.75, 1.0, 1.5 and 2×MIC (5 plates per compound) are prepared. The 48 hour culture is diluted 1:10 with SC-Leu, 2% glucose to OD_{600 nm} of 0.1. Plates from four 96 well dilute cultures are inoculated on one 384 well format using Biomek FX Workstation. Each plate contains 320 individually arrayed clones (four 96 well plates) and 2 rows of sterility controls. The clones are grown for 72 hours and read visually.

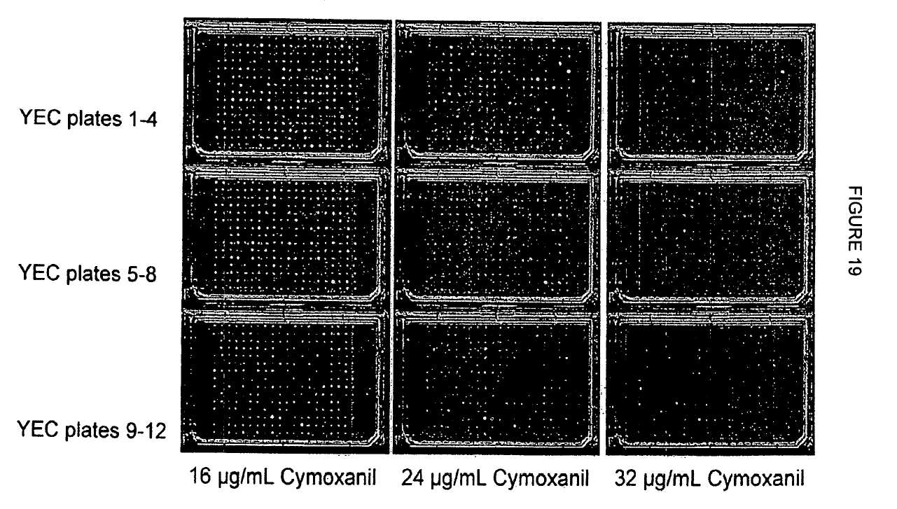

FIG. 19 shows a screen of 957 yeast clones against Fluconazole. The MIC was determined to be 32 μg/mL. The known target ER11 is seen growing in the upper left of the first plate.

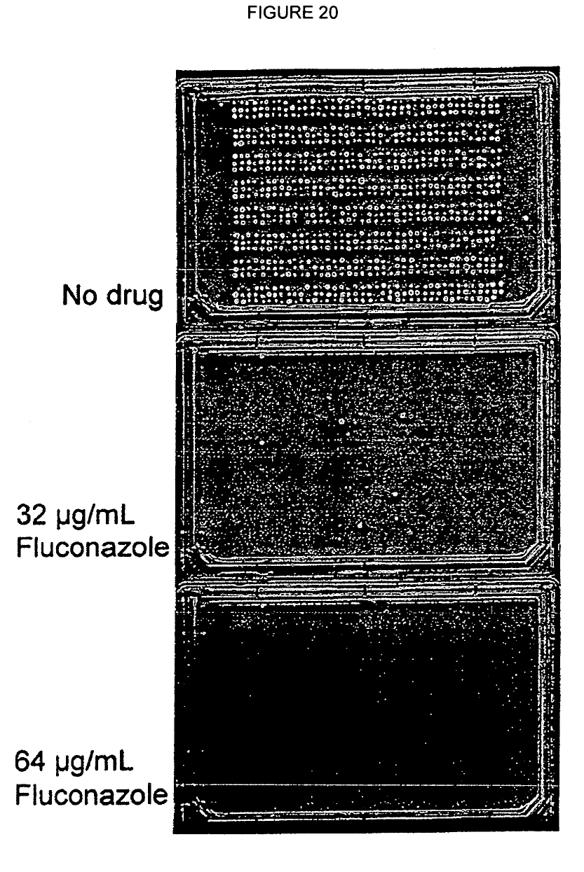

The system was optimized to a 1536 array of all 957 Yeast clones on one plate (FIG. 20). ERG11 is still growing growing as expected and the same clones that grew in the 384 array show up in this array. As such, it is now possible to run all clones on one plate.

While the present invention has been described with reference to what are presently considered to be the preferred examples, it is to be understood that the invention is not limited to the disclosed examples. To the contrary, the invention is intended to cover various modifications and equivalent arrangements included within the spirit and scope of the appended claims.

All publications, patents and patent applications are herein incorporated by reference in their entirety to the same extent as if each individual publication, patent or patent application was specifically and individually indicated to be incorporated by reference in its entirety. Where a term in the present application is found to be defined differently in a document incorporated herein by reference, the definition provided herein is to serve as the definition for the term.

### FULL CITATIONS FOR REFERENCES REFERRED TO IN THE SPECIFICATION

1. Marquardt, J. L., Siegele, D. A., Kolter, R., and Walsh, C. T. (1992) J Bacteriol 174, 5748-52.
2. Chen, P., and Bishai, W. R. (1998) Infect Immun 66, 5099-106.
3. Chen, D. Z., Patel, D. V., Hackbarth, C. J., Wang, W., Dreyer, G., Young, D. C., Margolis, P. S., Wu, C., Ni, Z. J., Trias, J., White, R. J., and Yuan, Z. (2000) Biochemistry 39, 1256-62.
4. Apfel, C. M., Locher, H., Evers, S., Takacs, B., Hubschwerlen, C., Pirson, W., Page, M. G., and Keck, W. (2001) Antimicrob Agents Chemother 45, 1058-64.
5. Miura, N., Kaneko, S., Hosoya, S., Furuchi, T., Miura, K., Kuge, S., and Naganuma, A. (1999) FEBS Lett 458, 215-8.
6. Calabrese, D., Bille, J., and Sanglard, D. (2000) Microbiology 146, 2743-54.
7. Osherov, N., Kontoyiannis, D. P., Romans, A., and May, G. S. (2001) J Antimicrob Chemother 48, 75-81.
8. Masuda, C. A., Xavier, M. A., Mattos, K. A., Galina, A., and Montero-Lomeli, M. (2001) J Biol Chem 276, 37794-37801.
9. Delling, U., Raymond, M., and Schurr, E. (1998) Antimicrob Agents Chemother 42, 1034-41.
10. Cotrim, P. C., Garrity, L. K., and Beverley, S. M. (1999) J Biol Chem 274, 37723-30.
11. Burger, H., Capello, A., Schenk, P. W., Stoter, G., Brouwer, J., and Nooter, K. (2000) Biochem Biophys Res Commun 269, 767-74.
12. Parr, A. L., Myers, T. G., Holbeck, S. L., Loh, Y. J.; and Allegra, C. J. (2001) Anticancer Drugs 12, 569-74.
13. Zhang, J. H., Chung, T. D., and Oldenburg, K. R. (1999) J Biomol Screen 4, 67-73.
14. Kobayashi et al. J. Bacteriology (2001) 183, 2646-2653.
15. Blakley, R. L. The Biochemistry of Folic Acid and Related Pteridines; North-Holland Publishing Co. Amsterdam, (1969); pp pp 219-358.
16. Huennekens, F. M. Adv Enzyme Regul (1994), 34, 397-419.
17. Olliaro, P. L.; Yuthavong, Y. Pharmacol Ther 1999), 81, 91-110.
18. Rollo, I. M. CRC Crit Rev Clin Lab Sci 1970, 1, 565-583.
19. Baccanari, D. P.; Kuyper, L. F. J Chemother (1993), 5, 393-399.
20. Huovinen, P. Clin Infect Dis (2001), 32, 1608-1614.
21. Casadaban, M. J., and Cohen, S. N. (1980) J Mol Biol 138, 179-207.
22. Lipinski, C. A. (2000) J Pharmacol Toxicol Methods 44, 235-49.
23. Kawabe, T., Fujihira, E., and Yamaguchi, A. (2000) J Biochem (Tokyo) 128, 195-200.
24. Nikaido, H., and Zgurskaya, H. I. (2001) J Mol Microbiol Biotechnol 3, 215-8.
25. Tai, N.; Ding, Y.; Schmitz, J. C.; Chu, E. Nucleic Acids Res (2002), 30, 4481-4488.
26. Hillcoat, B. L.; Nixon, P. F.; Blakley, R. L. Anal Biochem (1967), 21, 178-189.
27. Zhang, J. H.; Chung, T. D.; Oldenburg, K. R. J Biomol Screen (1999), 4, 67-73.
28. Stone, S. R.; Morrison, J. F. Biochim Biophys Acta (1986), 869, 275-285.
29. Baccanari, D. P.; Daluge, S.; King, R. W. Biochemistry (1982), 21, 5068-5075.
30. Chan, J. H.; Hong, J. S.; Kuyper, L. F.; Baccanari, D. P.; Joyner, S. S. et al. J Med Chem (1995), 38, 3608-3616.
31. Whitlow, M.; Howard, A. J.; Stewart, D.; Hardman, K. D.; Chan, J. H. et al. J Med Chem (2001), 44, 2928-2932.
32. Tang, P. C. M., McMahon, G.; Weinberger, H.; Kutscher, B.; App, H. Patent, (1998), WO 9850370.
33. Sawaya, M. R.; Kraut, J. Biochemistry (1997), 36, 586-603.
34. Guzman, L. M.; Belin, D.; Carson, M. J.; Beckwith, J. Tight regulation, modulation, and high-level expression by vectors containing the arabinose PBAD promoter. J Bacteriol (1995), 177, 4121-4130.

35. Morgan-Kiss, R. M.; Wadler, C.; Cronan, J. E., Jr. Long-term and homogeneous regulation of the Escherichia coli araBAD promoter by use of a lactose transporter of relaxed specificity. Proc Natl Acad Sci U S A (2002), 99, 7373-7377.

TABLE 1
DHFR inhibitors found to be competitive with dihydrofolate in Example 2

| | | IC₅₀ (nM) | IC₅₀ (nM) | |
|---|---|---|---|---|
| | | 3O μM | 100 μM | |
| Cpd # | Structure DHF | DHF | IR¹ | |
| 1 | 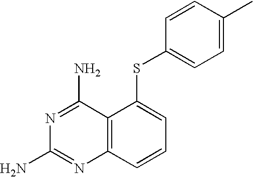 | 310 | 820 | 2.6 |
| 2 | 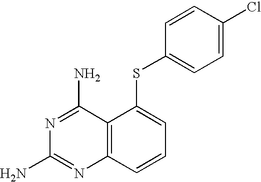 | 320 | 510 | 1.6 |
| 3 | 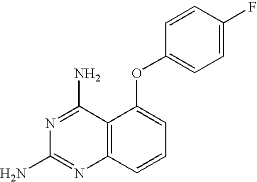 | 400 | 1.0 = 10³ | 2.6 |
| 4 | 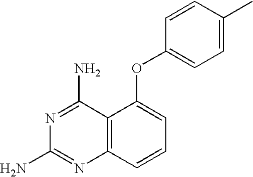 | 190 | 420 | 2.3 |
| 5 | 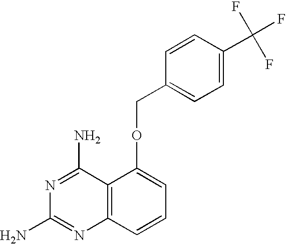 | 660 | 1.1 = 10³ | 1.7 |
| 6 | 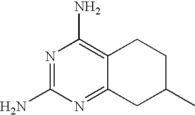 | 1.1 = 10⁴ | 2.4 =]10⁴ | 2.3 |
| 7 | 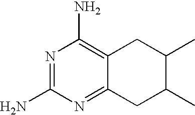 | 790 | 2.1 = 10³ | 2.6 |
| 8 | 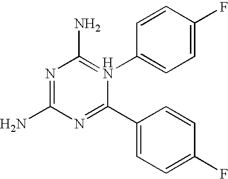 | 1.1 = 10⁴ | 1.6 = 10⁴ | 1.5 |
| 9 | 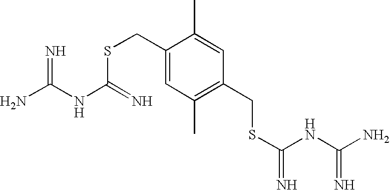 | 109 | 302 | 2.8 |
| 10 | 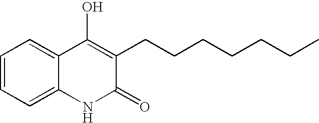 | 4.8 = 10⁴ | 1.2 = 10⁵ | 2.5 |
| 11 | 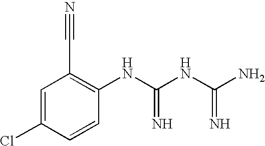 | 320 | 620 | 1.9 |

¹IC₅₀ ratio, (IC₅₀ in the presence of 100 μM DHF) / (IC₅₀ in the presence of 30 μM DHF).

TABLE 2
Analysis of resistant clones isolated
for known antibiotic-target pairs.

| | Fosfomycin | Trimethoprim | D-cycloserine |
|---|---|---|---|
| | selection for | selection for | selection for |
| | murA 320 μg/mL | folA 2.5 μg/mL | ddlA 160 μg/mL |
| Colonies | 7 | 20 | 8 |
| isolated | | | |
| PCR | 3/7 | 5/5 | 6/6 |
| verification¹ | | | |
| Sequence | 3/3 | 2/2 | 2/2 |
| verification² | | | |

¹PCR verification involved analytical amplification by PCR of the gene of interest from a mini-prep of plasmid DNA from resistant clones. Primer pairs used in that amplification were primers that annealed to the predicted target gene and to sequences flanking the cloning site of pGEM7. Shown are the number of positive clones and the number of clones tested.
²Sequence verification involved sequencing of a portion of the insert of the selected clone to determine if it contained the postulated gene. Shown are the number of positive clones and the number of clones tested.

TABLE 3
Growth inhbitory compounds from duplicate screens of 1000 molecules.

| | | Growth | Retest Minimum |
|---|---|---|---|
| Compound | | in Primary | Inhibitory |
| number | Stwcture | Screens | Concentration |
| BTB14887 | 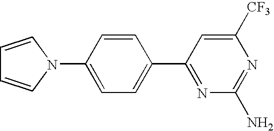 | 37, 30% | 125 μg/mL |
| SEW04978 | 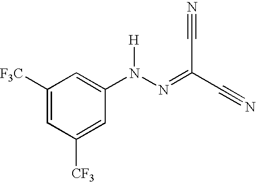 | 0.2, 0.02% | 32 μg/mL |
| SPB04137 | 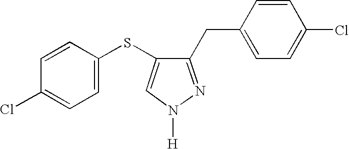 | 2.3, 2.1% | 500 μg/mL |
| RH00852 | 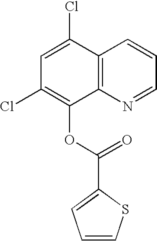 | 2.7, 2.8% | 16 μg/mL |
| RJF01047 | 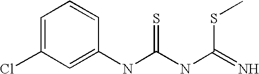 | 6.0, 12% | 125 μg/mL |

TABLE 4
Outcome of Selections for Multicopy Suppressions for
Controls and Two Test Compounds

| Compound | MIC^{a}(ug/ml) | Suppressor Clones^{b} |
|---|---|---|
| Fosfomycin | 80 | ispB-sfsB-murA-yrbA |
| Trimethoprim | 0.078 | yabF-kefC-folA |
| Cycloserine | 80 | (i) yaiW-yaiY-YaiZ-ddlA- |
| yaiB-phoA-psiF | | (ii) yaiY-YaiZ-ddlA-yaiB |
| | 16 | yabF-kefC-folA |
| 7 | | |
| | 4.0 | (i) yabF-kefC-folA |
| | | (ii) acrB |
| 4 | | |

^{a}Minimum growth inhibitory concentrations (E. coli MC1061/pGEM7) for the compounds indicated. Cells were grown on LB agar containing ampicillin (50 μg/mL) and streptomycin (50 μg/mL).
^{b}Indicated are the identities of complete open reading frames present in the cloning site of pGEM7 found by sequencing suppressor clones isolated from the random genomic library. In the case where two types of suppressor clones were identified they are listed (i) and (ii).

TABLE 5
Analogs of 7 and 4 and Their Respective Antibacterial
and Anti-DHFR Potencies as Determined in Example 5

| | R² | X | R¹ | MIC(μg/mL) | Kᵢ(nM) |
|---|---|---|---|---|---|
| 7 | CH₃ | — | — | 16 | 1000 |
| 6 | H | — | — | 64 | 1100 |
| 4 | — | O | p-CH₃ | 4.0 | 230 |
| 3 | — | O | p-F | 16 | 180 |
| 1 | — | S | p-CH₃ | 8.0 | 150 |
| 2 | — | S | p-Cl | 4.0 | 38 |
| Tmp | — | — | — | 0.03 | 3.0 |

1. A method of treating conditions that benefit from an inhibition of dihydrofolate reductase (DHFR), comprising administering to an animal in need thereof, an effective amount of compound selected from one or more of a compound of Formula I, and pharmaceutically acceptable salts and solvates thereof: 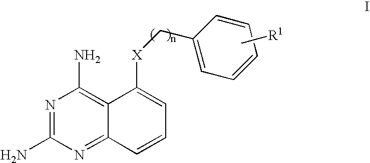
wherein
R¹ is selected from the group consisting of C₁₋₄alkyl, halo and CF₃;
X is O or S; and
n is 0 or 1.

2. The method according to claim 1, wherein the compound of Formula I is selected from one or more of compound 1, 2, 3, 4 and 5 as shown in Table 1, and pharmaceutically acceptable salts and solvates thereof.

3. A method of treating conditions that benefit from an inhibition of dihydrofolate reductase (DHFR), comprising administering to an animal in need thereof, an effective amount of compound selected from one or more of a compound of Formula II, and pharmaceutically acceptable salts and solvates thereof: 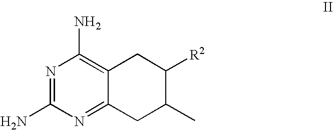
wherein R² is selected from the group consisting of H and C₁₋₄alkyl.

4. The method according to claim 3, wherein the compound of Formula II is selected from the group consisting of compound 6 and 7 as shown in Table 1, or pharmaceutically acceptable salts, solvates or hydrates thereof.

5. A method of treating conditions that benefit from an inhibition of DHFR, comprising administering to an animal in need thereof, an effective amount a compound selected from one or more of compounds 8 to 11 as shown in Table 1 and pharmaceutically acceptable salts and solvates thereof.

6. A method of treating bacterial infections comprising administering an effective amount of a compound selected from one or more of:
(a) a compound of Formula I, as defined in claim 1;
(b) a compound of Formula II, as defined in claim 3;
(c) compounds 8-11 as shown in Table 1; and
(d) pharmaceutically acceptable salts and solvates of (a), (b) and (c),
to a cell or animal in need thereof.

7. The method according to claim 6, wherein the bacterial infection is selected from an E. coli, Bacillus Subtilis, Streptococci, Staphylococci, Enterococci, Salmonella, Haemophilus influenza, mycobacterium spp, Pseudomonas aeruginosa, Bacillus anthracis and Helicobacter pylori infection.

8. The method according to claim 5, wherein the compound is selected from compound 9 as shown in Table 1 and pharmaceutically acceptable salts and solvates thereof.

9. A method of inhibiting DHFR in vitro comprising administering an effective amount of a compound selected from one or more of:
(a) a compound of Formula I, as defined in claim 1;
(b) a compound of Formula II, as defined in claim 3;
(c) a compound selected from compounds 8-11 as shown in Table 1; and
(d) salts and solvates of (a), (b) and (c),
to a cell or assay mixture.

10. The method according to claim 9, wherein the DHFR is bacterial DHFR.

11. The method according to claim 10, wherein the bacteria are E. coli, Bacillus Subtilis, Streptococci, Staphylococci, Enterococci, Salmonella, Haemophilus influenza, mycobacterium spp, Pseudomonas aeruginosa, Bacillus anthracis and Helicobacter pylori.

12. The method according to claim 11, wherein the bacteria are E. coli.

13. The method according to claim 9, wherein the compound is compound 9 as shown in table 1 and pharmaceutically acceptable salts and solvates thereof.

14. A method for identifying a candidate therapeutic agent and a cellular target molecule that is modulated by the agent comprising:
(a) contacting a plurality of test agents with a first target cell;
(b) selecting test agents from step (a) that inhibit the growth of the first target cell, wherein said selected test agents are candidate therapeutic agents;
(c) contacting a candidate therapeutic agent identified in step (b) with (i) the first target cell and separately with (ii) a second target cell that overexpresses one or more genes;
(d) comparing the growth of the first target cell with the second target cell wherein the inhibition of growth of the first target cell and not the second target cell indicates that the second target cell overexpresses the cellular target molecule of the candidate therapeutic; and
(e) identifying the cellular target molecule.

15. A method for identifying a candidate therapeutic agent and a cellular target molecule that is modulated by the agent comprising:
(a) contacting a candidate therapeutic agent with (i) a first target cell and separately with (ii) a second target cell that overexpresses one or more genes;
(b) comparing the growth of the first target cell with the second target cell wherein the inhibition of growth of the first target cell and not the second target cell indicates that the second target cell overexpresses the cellular target molecule of the candidate therapeutic; and
(c) identifying the cellular target molecule.

16. The method according to claim 14, wherein each test agent in step (a) is administered at a different concentration in order to determine a minimal inhibitory concentration (MIC) of each test agent and the MIC of each test agent is used step (c) when contacting the agent with the first and second target cells.

17. The method according to claim 16, wherein each test agent is contacted with the first and second target cells at one or more concentrations ranging from about 0.001 to about 50 times the MIC for that agent.

18. The method according to claim 17, wherein each test agent is contacted with the first and second target cells at one or more concentrations ranging from about 0.5 to about 40 times the MIC for that agent.

19. The method according to claim 14, wherein the second target cell is transformed with a multicopy random genomic library comprising all genes present in the first target cell.

20. The method according to claim 15, wherein the second target cell is transformed with a multicopy random genomic library comprising all genes present in the first target cell.

21. The method according to claim 19, wherein identification of the cellular target molecule being overexpressed in the second target cell is carried out by obtaining a sample comprising the DNA from the second target cell and determining the sequence of the target molecule being overexpressed.

22. The method according to claim 19, wherein the sequence of the target molecule is determined using PCR.

23. The method according to claim 14, wherein the second target cell comprises one or more cell types, each overexpressing at least an open reading frame from one gene in the genome of an organism from which the cell is taken.

24. The method according to claim 23, wherein the second target cell comprises a plurality of cell types, each overexpressing at least an open reading from genes that are essential for the growth of the organism.

25. The method according to claim 23, wherein the second target cell comprises a plurality of cell types, each overexpressing at least an open reading from genes encoding specific proteins of interest.

26. The method according to claim 23, wherein the one or more cell types are pooled and the candidate therapeutic agent is contacted with the pool of one or more cell types.

27. The method according to claim 26, wherein the identification of the gene being overexpressed in the one or more cell types is done using hybridization analysis with an ordered microarray of the plasmid DNA from the organism.

28. The method according to claim 14, wherein the second target cell comprises one or more cell types which are in the form of an ordered library of cells comprising at least an open reading frame of genes from an organism from which the cell is taken.

29. The method according to claim 28, wherein the genes are those which are essential to growth of the organism.

30. The method according to claim 28, wherein the growth of each type of cell in the ordered library of second target cells is compared to the growth of the first target cell, wherein inhibition of growth of the first target cell and not the second target cell type indicates that the second target cell type overexpresses the cellular target molecule of the candidate therapeutic agent.

31. The method according to claim 30, wherein the ordered library of cell types, each over-expressing a unique gene from the organism, is in the form of microarray.

32. The method according to claim 26, wherein the pool of second target cells comprises cells that do not overexpress genes encoding an efflux pump.

33. The method according to claim 28, wherein the second target cell comprises cells that do not overexpress genes encoding an efflux pump

34. The method according to claim 14, wherein the second target cells comprises cells overexpressing at least the open reading frame of drug transport or efflux pump genes for the organism.

35. The method according to claim 34, wherein identification of a drug transport or efflux pump as a cellular target molecule for the candidate therapeutic agent indicates cellular resistance to the candidate therapeutic agent.

36. The method according to claim 14, wherein wherein the first and second target cells are selected from bacterial, fungus, parasites, yeasts and cancer cells.

37. The method according to claim 36, wherein the bacterial cells are selected from the group consisting of E. coli, Bacillus subtilis, Streptococci, Staphylococci, Enterococci, Salmonella, Haemophilus influenza, mycobacterium spp, Pseudomonas aeruginosa, Bacillus anthracis and Helicobacter pylori.

38. The method according to claim 36, wherein the yeast cells are from Saccaraomyces cerevisiae.

39. A kit for use in identifying a therapeutic agent and its cellular target comprising a first target cell to which one wishes to generate a therapeutic agent and a second target cell that overexpresses one or more genes present in the first target cell.

41. A method of conducting a drug discovery business comprising:
(a) providing one or more assay systems for identifying a potential therapeutic agent based on the method according to claim 14;
(b) conducting therapeutic profiling of agents identified in step (a), or further analogs thereof, for efficacy and toxicity in animals; and
(c) formulating a pharmaceutical preparation including one or more agents identified in step (b) as having an acceptable therapeutic profile.

42. The method at claim 14 wherein the cellular target molecule is isolated.

43. The method at claim 15 wherein the cellular target molecule is isolated.
